(19) United States
(12) Patent Application Publication
(10) Pub. No.: US 2011/0236895 A1
(43) Pub. Date: Sep. 29, 2011

(54) METHOD FOR PREPARING SAMPLE, SOLUTION FOR PREPARING SAMPLE AND STOOL COLLECTION KIT METHOD FOR ANALYZING A NUCLEIC ACID

(75) Inventors: Yasuo Tanigami, Tokyo (JP); Tomonori Nagaoka, Tokyo (JP)

(73) Assignee: OLYMPUS CORPORATION, Tokyo (JP)

(21) Appl. No.: 13/153,045

(22) Filed: Jun. 3, 2011

Related U.S. Application Data
(63) Continuation of application No. PCT/JP2009/070171, filed on Dec. 1, 2009.

(30) Foreign Application Priority Data
Dec. 5, 2008 (JP) P2008-310989
Dec. 5, 2008 (JP) P2008-310990

(51) Int. Cl. C12Q 1/68; C07H 1/08; C09K 15/22; C09K 15/20; C09K 15/06

(52) U.S. Cl. 435/6.11; 435/6.1; 435/6.18; 536/25.41; 435/6.12; 536/25.42; 252/397; 252/403; 252/407

(57) ABSTRACT

The present invention relates to the providing of a method for preparing a sample from a nucleic acid-containing sample such as biological samples, where inhibitory substance's action against a enzyme reaction using a nucleic acid as substrate are decreased, a solution for preparing a sample used for the method, a stool collection kit used in that method, and a method for recovering and analyzing a nucleic acid in a nucleic acid-containing sample using a sample prepared using the preparation method of the present invention. A method for preparing a sample according to the present invention is a method for preparing a sample being used for analyzing a nucleic acid, and is characterized in that a nucleic acid-containing sample is mixed with a solution having one or more members selected from the group consisting of a polycation and a chelating agent as an active ingredient.

The present continuation application is on PCT International Patent Application No. PCT/JP2009/070171, which claims priority on the basis of Japanese Patent Application No. 2008-310989, filed in Japan on Dec. 5, 2008, and of Japanese Patent Application No. 2008-310990, filed in Japan on Dec. 5, 2008, the contents of which are incorporated herein by reference.

### TECHNICAL FIELD

The present invention relates to a method for preparing a sample in order to recover a nucleic acid contained in a nucleic acid-containing sample, a solution for preparation a sample, a prepared sample prepared by the above preparation method, a method for recovering a nucleic acid from the prepared sample, and a method for analyzing nucleic acid that uses a nucleic acid recovered by using the above nucleic acid recovering method.

### BACKGROUND ART

The number of colorectal cancer patients is currently continuing to increase rapidly each year in not only the U.S. and Europe, but in Japan as well, and is becoming one of the leading causes of cancer-related deaths. This is thought to be due to the growing proliferation of a Western style diet consisting primarily of red meat among the Japanese people. More specifically, roughly 60,000 persons are afflicted with colorectal cancer each year, and in terms of the number of deaths by organ, colorectal cancer is ranked third after gastric cancer and lung cancer, and is predicted to continue to increase in the future. On the other hand, differing from other forms of cancer, colorectal cancer has a nearly 100% cure rate if treated soon after onset. Thus, it is extremely significant to include colorectal cancer in early cancer screening examinations, and research and development of testing methods for early discovery of colorectal cancer is proceeding at a rapid pace.

Methods such as barium enema examinations and colonoscopies are performed as testing methods for early discovery of colorectal cancer. Barium enema examinations consist of injecting barium into the large intestine and allowing it to adhere to the mucosal membranes of the large intestine, irradiating the intestine with X-rays to capture images of any surface irregularities, and then observing the surface. On the other hand, colonoscopy consists of observing the inside of the large intestine directly with an endoscope. Colonoscopy in particular enables high levels of sensitivity and specificity, while also offering the advantage of allowing the excision of polyps and early forms of cancer.

However, in addition to be associated with high costs, these examinations place a considerable burden on the patient while also having the problem of being accompanied by complication risks. For example, barium enemas have risks associated with X-ray exposure and intestinal obstruction. In addition, colonoscopy is an invasive procedure since the endoscope is inserted directly into the large intestine. Moreover, the endoscopic procedure requires an experienced technician and the number of facilities where this examination can be performed is limited. Consequently, these examinations are not suitable for colorectal cancer examinations targeted at asymptomatic, healthy individuals as part of routine health examinations and the like.

In recent years, fecal occult blood tests have been widely performed as a non-invasive and inexpensive method for primary screening for colorectal cancer. The fecal occult blood test is a test for the presence of hemoglobin originating in erythrocytes contained in fecal matter, and is used as a method for indirectly predicting the presence of colorectal cancer. Factors behind the widespread use of the fecal occult blood test include stool samples being able to be collected and stored at room temperature eliminating the need for refrigerators, freezers and other special storage conditions, samples being able to be collected easily at home, and the test procedure being extremely simple. However, since the fecal occult blood test has low sensitivity of only about 25%, it has the problem of a high percentage of colorectal cancer being overlooked. Moreover, it also has a low positive predictive value, with the percentage of actual colorectal cancer patients among subjects judged to be positive in the fecal occult blood test being only about 10%, thus resulting in a large number of false positives. Consequently, there is a strong need for the development of a new examination method offering higher reliability.

Recent development in gene analysis technology has caused some attempts to analyze nucleic acids in biological sample in order to help diagnose diseases, treatments and the like. Because of using nucleic acids present in biological samples such as stool and blood, the nucleic acid analysis has advantages of being non-invasive and a lighter burden for patients compared to the other clinical tests such as colonoscopy, and is suitable for routine health examinations or the like. In addition, since genes correlating to diseases are directly investigated, the nucleic acid analysis also has an advantage of obtaining highly reliable results. For example, the method using nucleic acid analysis to investigate the presence of cancer cells or cancer cell-derived genes directly is considered to be more reliable than the fecal occult blood test, which tests for the presence of blood from the digestive tract that occurs indirectly accompanying the onset of colorectal cancer. Furthermore nucleic acid analysis can be used for early detection and stage of cancers.

In order to accurately detect cancer cells and the like in stool samples, it is important to efficiently recover cancer cell-derived nucleic acids from those stool samples. In particular, since cancer cell-derived nucleic acids are only present in trace amounts in stool samples, and stool samples also contain large amounts of digestive remnants and bacteria, nucleic acids are decomposed extremely easily. Consequently, in order to efficiently recover nucleic acids, and particularly nucleic acids derived from mammalian cells such as human cells, from stool samples, it is important to prevent decomposition of nucleic acids within the stool and prepare the stool sample so that it can be stored stably until the time of the testing procedure. An example of such a stool sample processing method consists of separating cancer cells that have exfoliated from the large intestine or other constituent of the digestive tract from a collected stool sample. Separation of cancer cells from stool makes it possible to inhibit the effects of bacterial proteases, DNase, RNasc and other degrading enzymes. Examples of methods that have been disclosed for separating cancer cells from stool include: (1) a method for separating cells from stool, comprising: (a) a step for cooling the stool to a temperature below its gel freezing point, and (b) a step for collecting cells from the stool while maintaining at a temperature below the gel freezing point so that the stool substantially remains completely intact (see, for example, Japanese Translation of PCT Application No. H11-511982). Another example of such a method consists of: (2) dispersing the stool in a transport medium containing a protease inhibitor, mucous dissolver and bactericide at a normal ambient temperature, followed by isolating the colorectal exfoliated cells (see, for example, Japanese Translation of PCT Application No. 2004-519202).

On the other hand, numerous fixation methods, such as formalin fixation or alcohol fixation, have conventionally been employed to maintain the morphology of collected cells until the time of observation in cases of histological and cytological observation of cell morphology. A method that has been disclosed as an example of a method that applies these fixation methods consists of (3) a cell solution preservative comprising an alcohol that is miscible with an amount of water sufficient for colonizing mammalian cells, an amount of anti-aggregation agent sufficient for preventing aggregation of mammalian cells in the solution, and a buffer for maintaining the pH of the solution within a range of 4 to 7 during the time the cells are stored, which is used as a storage solution for enabling mammalian cell samples to be stored for long periods of time or enable cells to be observed following storage (see, for example, Patent Japanese Unexamined Patent Application, First Publication No. 2003-153688).

In addition, disclosed examples of storage solutions that enable histological and cytological examinations of cells as well as molecular analyses of proteins or nucleic acids and the like present in cells after storage include (4) a universal collection medium containing a buffer component, at least one alcohol component, a fixative component and a chemical agent that inhibits decomposition of at least one member selected from the group consisting of RNA, DNA and protein (see, for example, Japanese Translation of PCT Application No. 2004-500897), and a non-aqueous solution containing 5 to 20% polyethylene glycol and 80 to 95% methanol (see, for example, Japanese Translation of PCT Application No. 2005-532824). In addition, (6) a method for stably storing nucleic acids in biological sample, mainly whole blood sample, comprising: extracting nucleic acids promptly from cells contained in a collected biological sample, and adding at least a single type of inhibitory agent on genetic induction into the extract to inhibit genetic induction outside a cell, thereby stabilizing nucleic acids (see, for example, Japanese Translation of PCT Application No. 2004-534731). Examples of the inhibitory agent on genetic induction indicated in the document include a cationic compound, detergent, especially cationic detergent, a chaotropic salt, an inhibitory agent on ribonuclease, a chelating agent, an organic solvent, and an organic reducing agent.

On the other hand, since biological samples such as stool and blood contain various kinds of components other than nucleic acids, in the case of recovering nucleic acids from these biological samples, it is often difficult to recover enough highly purified nucleic acids because of carryover of large amounts of contaminants. In particular, stool or the like contains substances such as bile acids and salts thereof, which have an inhibitory action against nucleic acid chain elongation reactions such as polymerase chain reaction (PCR) (see, for example, Wilson, I. G., Applied and Environmental Microbiology, 1997, Vol. 63, pp. 3741-3751). Biological samples have only trace amounts of nucleic acids and nucleic acids recovered from biological samples are generally analyzed by using nucleic acid chain elongation reactions such as PCR. But in the case of using nucleic acids recovered from stool or the like, the method has a shortcoming of reduction in accuracy and sensitivity of the examination due to inhibitory substances derived from biological samples.

Consequently, there are some methods disclosed which are for inhibiting the carryover of inhibitory substances in extracting and purifying nucleic acids from biological samples such as stool. Examples of methods that have been disclosed include: (7) a method for purifying, fixing, and/or separating nucleic acids from a material sample, comprising: adding the buffer to a sample containing nucleic acids, wherein the buffer fulfills at least one of the following conditions: the pH value being within a range of 2 to 7, the salt concentration being at least 100 mM, and neutralized phenol contained, thereby recovering highly purified nucleic acids which contain only small amounts of inhibitory substances or the like (see, for example, Japanese Translation of PCT Application No. 2003-521250). In addition, another example of such a method that has been disclosed is a method for enzymatic nucleic acid amplification reaction by using an enzyme which comprises washing a material being tested with an organic solvent to eliminate inhibitory substances on nucleic acid amplification reaction by using an enzyme, followed by amplifying enzymatically nucleic acids derived from cells contained in the material being tested (see, for example, PCT international publication No. WO 00/08136).

Furthermore, the examples also include: (9) a nucleic acid synthesizing method for amplifying the target nucleic acid in a sample characterized in previously contacting the sample to insoluble high-molecular compounds such as polyanion (high-molecular compound having a repeated structure containing anion) and salts thereof (sec, for example, Japanese Unexamined Patent Application, First Publication No. 2001-258562), and (10) a method for eliminating at least a single type of contaminants, comprising: a step of contacting a sample containing nucleic acids to at least a single type of aggregating agent and forming aggregated precipitates, followed by a step of separating nucleic acids from the aggregated precipitates (see, for example, Japanese Translation of PCT Application No. 2008-500066).

### SUMMARY OF THE INVENTION

As a result of intensive and extensive studies in order to solve the above-mentioned problems, the inventors of the present invention found that when a nucleic acid-containing sample is mixed with a solution for preparing a sample having one or more members selected from the group consisting of a polycation and a chelating agent as an active ingredient thereof prior to a step of extracting nucleic acids, it is able to decrease the inhibitory actions against enzyme reactions using a nucleic aid as a substrate by inhibitory substances contained in a nucleic acid-containing sample, thereby leading to completion of the present invention.

Namely, the present invention includes the following aspects.

(1) A method for preparing a sample, comprising: mixing a nucleic acid-containing sample with a solution for preparing a sample having one or more members selected from the group consisting of a polycation and a chelating agent as an active ingredient, wherein the prepared sample is used for recovering a nucleic acid.

(2) In the method for preparing a sample according to the aspect (1), it is preferred that the solution for preparing a sample further contain a water-soluble organic solvent as an active ingredient.

(3) In the method for preparing a sample according to the aspect (1) or (2), it is preferred that the solution for preparing a sample contain a polylysine as a polycation.

(4) In the method for preparing a sample according to the aspect (1) or (2), it is preferred that the solution for preparing a sample contain EDTA as a chelating agent.

(5) In the method for preparing a sample according to any one of the aspects (1) to (4), it is preferred that the solution for preparing a sample have a buffering action.

(6) In the method for preparing a sample according to any one of the aspects (1) to (5), it is preferred that the pH of the solution for preparing a sample be from 2 to 6.5.

(7) In the method for preparing a sample according to any one of the aspects (2) to (6), it is preferred that the water-soluble organic solvent be one or more members selected from the group consisting of a water-soluble alcohol, ketone and aldehyde.

(8) In the method for preparing a sample according to any one of the aspects (2) to (6), it is preferred that the water-soluble organic solvent be one or more members selected from the group consisting of a water-soluble alcohol and ketone, and that the concentration of the water-soluble organic solvent is 30% or more.

(9) In the method for preparing a sample according to the aspect (7) or (8), it is preferred that the water-soluble organic solvent contain one or more members selected from the group consisting of ethanol, propanol and methanol as water-soluble alcohol.

(10) In the method for preparing a sample according to any one of the aspects (2) to (8), it is preferred that the water-soluble organic solvent be ethanol.

(11) In the method for preparing a sample according to any one of the aspects (7) to (9), it is preferred that the water-soluble organic solvent contain one or more members selected from the group consisting of acetone and methyl ethyl ketone as ketone.

(12) In the method for preparing a sample according to any one of the aspects (2) to (6), it is preferred that the water-soluble organic solvent be an aldehyde, and the concentration of the water-soluble organic solvent is within a range of 0.01 to 30%.

(13) In the method for preparing a sample according to any one of the aspects (1) to (12), it is preferred that in terms of a mixing ratio of the nucleic acid-containing sample and the solution for preparing a sample, a volume of the solution for preparing the sample be one or more relative to 1 volume of the nucleic acid-containing sample.

(14) In the method for preparing a sample according to any one of the aspects (1) to (13), it is preferred that the mixture of the nucleic acid-containing sample and the solution for preparing a sample be stored for a predetermined amount of time.

(15) In the method for preparing a sample according to the aspect (14), it is preferred that the amount of time during which the mixture is stored be 1 hour or more.

(16) In the method for preparing a sample according to the aspect (14), it is preferred that the amount of time during which the mixture is stored be 12 hours or more.

(17) In the method for preparing a sample according to the aspect (14), it is preferred that the amount of time during which the mixture is stored be 24 hours or more.

(18) In the method for preparing a sample according to the aspect (14), it is preferred that the amount of time during which the mixture is stored be 72 hours or more.

(19) In the method for preparing a sample according to any one of the aspects (6) to (18), it is preferred that the pH of the solution for preparing a sample be from 3 to 6.

(20) In the method for preparing a sample according to any one of the aspects (6) to (18), it is preferred that the pH of the solution for preparing a sample be from 4.5 to 5.5.

(21) In the method for preparing a sample according to any one of the aspects (1) to (20), it is preferred that the solution for preparing a sample further contain a surface active agent.

(22) In the method for preparing a sample according to any one of the aspects (1) to (21), it is preferred that the solution for preparing a sample further contain a colorant.

(23) In the method for preparing a sample according to any one of the aspects (1) to (22), it is preferred that the nucleic acid-containing sample be one or more members selected from the group consisting of stool, blood, and urine.

(24) In the method for preparing a sample according to any one of the aspects (1) to (22), it is preferred that the nucleic acid-containing sample be stool.

(25) A solution for preparing a sample, comprising: one or more members selected from the group consisting of a polycation and a chelating agent as an active ingredient, wherein the solution is used for recovering a nucleic acid from the nucleic acid-containing sample.

(26) In the solution for preparing a sample according to the aspect (25), it is preferred that the solution for preparing a sample contain a polylysine as a polycation.

(27) In the solution for preparing a sample according to the aspect (25), it is preferred that the solution for preparing a sample contain EDTA as a chelating agent.

(28) In the solution for preparing a sample according to any one of the aspects (25) to (27), it is preferred that the solution for preparing a sample further contain a water-soluble organic solvent as an active ingredient.

(29) In the solution for preparing a sample according to the aspect (28), it is preferred that the water-soluble organic solvent be one or more members selected from the group consisting of a water-soluble alcohol, ketone and aldehyde.

(30) A stool collection kit, comprising: a stool collection container; and a solution for preparing a sample having one or more members selected from the group consisting of a polycation and a chelating agent as an active ingredient, wherein the stool collection container includes the solution for preparing a sample.

(31) A prepared sample prepared by the method for preparing a sample according to any one of the aspects (1) to (24).

(32) A method for analyzing a nucleic acid comprising:

(i) a step for preparing a prepared sample by mixing a nucleic acid-containing sample with a solution for preparing a sample having one or more members selected from the group consisting of a polycation and a chelating agent as an active ingredient,

(ii) a step for recovering a nucleic acid from a cell contained in the prepared sample prepared in the step (i), and

(iii) a step for analyzing the nucleic acid recovered in the step (ii).

(33) In the method for analyzing a nucleic acid according to the aspect (32), it is preferred that the step (i) include:

(i-1) a step for immersing the nucleic acid-containing sample in the solution for preparing a sample, or

(i-2) a step for immersing the nucleic acid-containing sample in the solution for preparing a sample, and mixing to suspend the nucleic acid-containing sample.

(34) A method for analyzing a nucleic acid, comprising: conducting reverse transcriptase reaction or nucleic acid chain elongation reaction in a reaction solution containing a polycation.

(35) In the method for analyzing a nucleic acid according to the aspect (34), it is preferred that the reaction solution contain a polylysine as a polycation.

(36) A method for recovering nucleic acid from a stool, comprising: simultaneously recovering a nucleic acid derived from indigenous intestinal bacterium and a nucleic acid derived from an organism other than indigenous intestinal bacterium, from the stool sample, and the stool sample is prepared by mixing a collected stool with a solution for preparing a sample having one or more members selected from the group consisting of a polycation and a chelating agent as an active ingredient.

(37) In the method for recovering a nucleic acid from a stool according to the aspect (36), it is preferred that the nucleic acid derived from the organism other than indigenous intestinal bacterium be the nucleic acid derived from a mammalian cell.

(38) In the method for recovering a nucleic acid from a stool according to the aspect (36) or (37), it is preferred that the step for recovering a nucleic acid include:

(a) a step for denaturing a protein in the stool sample and thereby extracting a nucleic acid from indigenous intestinal bacterium and an organism other than indigenous intestinal bacterium in the stool sample; and

(b) a step for recovering the nucleic acid extracted in the step (a).

(39) In the method for recovering a nucleic acid from a stool according to the aspect (38), it is preferred that the step for recovering a nucleic acid include further, following the step (a) and prior to the step (b),

(c) a step for removing the protein denatured in the step (a).

(40) In the method for recovering a nucleic acid from a stool according to the aspect (38) or (39), it is preferred that denaturing of a protein in the step (a) be carried out using one or more material selected from the group consisting of a chaotropic salt, an organic solvent and a surface active agent.

(41) In the method for recovering a nucleic acid from a stool according to the aspect (40), it is preferred that the organic solvent be phenol.

(42) In the method for recovering a nucleic acid from a stool according to any one of the aspects (39) to (41), it is preferred that the removal of denatured protein in the step (c) be carried out using chloroform.

(43) In the method for recovering a nucleic acid from a stool according to any one of the aspects (38) to (42), it is preferred that the recovery of nucleic acid in the step (b) include:

(b1) a step for adsorbing the nucleic acid extracted in the step (a) to an inorganic support, and

(b2) a step for eluting the nucleic acid adsorbed in the step (b1) from the inorganic support.

(44) In the method for recovering a nucleic acid from a stool according to any one of the aspects (38) to (43), it is preferred that the step for recovering a nucleic acid include further, prior to the step (b),

(d) a step for recovering a solid component from the stool sample.

(45) A method for analyzing a nucleic acid comprising: conducting an analysis of a nucleic acid derived from a mammalian cell, wherein the nucleic acid is recovered from a stool sample by use of the method for recovering a nucleic acid according to any one of the aspects (38) to (44).

(46) In the method for analyzing a nucleic acid according to the aspect (45), it is preferred that the mammalian cell be a gastrointestinal tract cell.

(47) In the method for analyzing a nucleic acid according to the aspect (45), it is preferred that the mammalian cell be a cell exfoliated from a large intestine.

(48) In the method for analyzing a nucleic acid according to any one of the aspects (45) to (47), it is preferred that the nucleic acid derived from a mammalian cell be a marker indicating a neoplastic transformation.

(49) In the method for analyzing a nucleic acid according to any one of the aspects (45) to (47), it is preferred that the nucleic acid derived from a mammalian cell be a marker indicating an inflammatory gastrointestinal disease.

(50) In the method for analyzing a nucleic acid according to any one of the aspects (45) to (47), it is preferred that the nucleic acid derived from a mammalian cell be a nucleic acid derived from COX-2 gene.

(51) In the method for analyzing a nucleic acid according to any one of the aspects (45) to (50), it is preferred that the analysis be one or more of RNA analysis and DNA analysis.

(52) In the method for analyzing a nucleic acid according to the aspect (51), it is preferred that the RNA analysis be one or more analysis selected from the group consisting of an analysis for insertion, deletion, substitution, duplication or inversion of one or more bases in the RNA, an analysis for a splicing variant, an mRNA expression analysis, and a functional RNA analysis.

(53) In the method for analyzing a nucleic acid according to the aspect (51), it is preferred that the DNA analysis be one or more of a mutation analysis and an analysis of an epigenetic change.

(54) In the method for analyzing a nucleic acid according to the aspect (53), it is preferred that the mutation analysis be an analysis for one or more mutations of an insertion, deletion, substitution, duplication or inversion of one or more bases.

(55) In the method for analyzing a nucleic acid according to the aspect (53), it is preferred that the analysis of an epigenetic change be one or more of a DNA methylation analysis and a DNA demethylation analysis.

(56) In the method for analyzing a nucleic acid according to the aspect (53), it is preferred that the mutation analysis be a mutation analysis of a K-ras gene.

### BRIEF DESCRIPTION OF THE DRAWINGS

FIG. 1 is a diagram showing an embodiment of a stool collection container which can be used for a stool collection kit according to the present invention.

FIG. 2 is a diagram showing an embodiment of a stool collection container which can be used for a stool collection kit according to the present invention.

FIG. 3 is a graph showing the results of a relative comparison of expressed amounts of GAPDH gene in RNA derived from Stool Samples 1-1 to 1-8 in Example 1.

FIG. 4 is a graph showing the results of a relative comparison of expressed amounts of GAPDH gene in RNA derived from Nucleic Acid Samples 2-1 to 2-8 in Example 2.

FIG. 5 is a graph showing the results of a relative comparison of expressed amounts of GAPDH gene in RNA derived from Nucleic Acid Samples 3-1 to 3-8 in Example 3.

FIG. 6 is a graph showing the results of a relative comparison of expressed amounts of GAPDH gene in RNA derived from Stool Samples 5-1 to 5-4 in Example 5.

FIG. 7 is a graph showing amounts of RNA recovered from each of the stool samples in Reference Example 1.

FIG. 8 is a graph showing amounts of RNA recovered from stool samples prepared using ethanol solutions of various concentrations in Reference Example 3.

### BEST MODE FOR CARRYING OUT THE INVENTION

In the present invention and in the present description of the present application, the term “%” refers to “% by volume (vol %)”, unless otherwise specified.

In the present invention, the term a “inhibitory substance” refers to a substance that has an inhibitory action against an enzyme reaction using a nucleic acid as a substrate. The enzyme reaction is not particularly limited as long as it is an enzyme reaction using a nucleic acid as a substrate, and examples of the reaction include enzyme reactions generally used in nucleic acid analysis such as a reverse transcriptase and a nucleic acid chain elongation reaction. In the present description, the term a “nucleic acid chain elongation reaction” refers to a reaction of elongating a nucleic acid chain by a polymerase or a ligase. Examples of nucleic acid chain elongation reactions by polymerases include PCR, realtime-PCR, SDA (Standard Displacement Amplification) and the like. Examples of nucleic acid chain elongation reactions by ligases include LCR (Ligase Chain Reaction) and the like. In particular, the reaction is preferably an amplification reaction that is accompanied by nucleic acid chain elongation by DNA polymerase such as PCR.

### <A Method for Preparing a Sample>

A method for preparing a sample according to the present invention is a method for preparing a sample from a nucleic acid-containing sample, wherein the prepared sample is used for recovering a nucleic acid. The method for preparing a sample according to the present invention is characterized in that a nucleic acid-containing sample is mixed with a solution for preparing a sample having one or more members selected from the group consisting of a polycation and a chelating agent as an active ingredient of the solution. It is enabled to efficiently decrease the inhibitory action of inhibitory substances by mixing a nucleic acid-containing sample with a solution having a polycation and/or a chelating agent as an active ingredient thereof.

Consequently, by recovering a nucleic acid from the prepared sample prepared by using the method for preparing a sample according to the present invention (to also be referred to as the “prepared sample of the present invention”) and analyzing the recovered nucleic acid, it is able to decrease the effects of inhibitory substances and the like, thereby obtaining more highly reliable analysis results.

In the present invention, the nucleic acid-containing sample is not particularly limited as long as it is a sample containing a nucleic acid. It may be a biological sample or a sample collected from nonliving materials. Examples of biological samples include stool, urine, bone marrow fluid, lymph fluid, expectorated sputum, sperm, bile, pancreatic fluid, ascetic fluid, exudate fluid, amnion fluid, intestinal lavage fluid, lung lavage fluid, bronchial lavage fluid, and bladder lavage fluid. Although the biological sample is not particularly limited as long as it is a sample collected from an organism, it is preferably a sample collected from a mammal, is more preferably a sample collected from a human being. Furthermore, it may be a culture of a cultured cell and the like. Examples of samples collected from nonliving materials include water samples collected from soil, sea, river, lake, and the like. Since soil and the like contain nucleic acids derived from microbial organisms.

The nucleic acid-containing sample supplied for the method for preparing a sample according to the present invention, is preferably stool, urine, or blood, and is more preferably stool.

In this invention, the nucleic acid-containing sample may be one which has been stored for a certain period of time following the collection thereof, but is preferably one which has just been collected. In addition, the nucleic acid-containing sample may be one which has been untreated from the collection thereof or one treated in some way. The method for treating is not particularly limited as long as the method does not decompose nucleic acids contained in a nucleic acid-containing sample, and a treating method which is generally conducted on a biological sample and the like can be used. Examples of the treating methods include washing to remove mucosal fluid and the like, dilution with normal saline solution and the like, and separation or condensation of cellular components.

In addition, the stool supplied for the method for preparing a sample according to the present invention, is not particularly limited as long as it originates from an animal (a subject), but is preferably one that originates from a mammal, and is more preferably one that originates from a human being. For example, the stool supplied is preferably a stool of a human being collected for routine health examinations, a diagnosis or the like, but it may also be a stool from livestock wild animal, or the like. In addition, the stool may be one which has been stored for a certain period of time following the collection thereof, but is preferably one which has just been collected. Furthermore, the stool is preferably collected immediately after the excretion thereof, but may be collected after a certain period of time following the excretion thereof.

The solution for preparing a sample used in the method for preparing a sample according to the present invention (to also be referred to as the “solution for preparing of the present invention”) contains one or more members selected from the group consisting of a polycation and a chelating agent as an active ingredient of the solution.

Although, concrete functional mechanism of the effect of decreasing inhibitory action achieved by polycation is still not understood, it is speculated that polycation interact directly with inhibitory substances, thereby the inhibitory action thereof decreased.

In the present invention and the present description, the term “polycation” refers to a high-molecular compound having a repeated structure containing anion or salts thereof. Examples of anion include an amino group. More specifically, Examples of polycation include a polypeptide having an anion group in a side chain such as polylysine as shown by the following formula (I), and a polymer synthesized by polymerizing a monomer having an anion group in side chain such as polyacrylamide. Note that these polypeptides and polymers are only required to be electrically-positive in the whole compound, and they do not need to have an anion group in each side chain of a repeating unit (such as an amino acid and a monomer). It is preferred that the high-molecular compound having a repeated structure containing anion preferably have an anion group in a side chain of every repeating unit. More specifically, examples of the polycation include, adding to polylysine and polyacrylamide, polyvinylamine, polyacrylamine, polyethylamine, polymethacrylamine, polyvinylmethylimidazole, polyvinylpyridine, polyarginine, chitosan, 1,5-dimethyl-1,5-diazaundecamethylene-polymethobromide, poly(2-dimethylaminoethyl (meth) acryrate), poly(2-diethylaminoethyl(meth)acryrate), poly(2-trimethylammoniumethyl(meth)acryrate), polydimethylaminomethylstyrene, polytrimethylammoniummethylstyrene, polyomithine and polyhistidine. In the present invention, the polycation is preferably polylysine or polyacrylamide, and is more preferably polylysine. The solution for preparing of the present invention may contain only a single type of polycation or may contain two or more types of polycations.

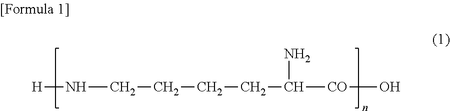

The concentration of a polycation in the solution for preparing of the present invention is not particularly limited as long as it is a concentration capable of decreasing an inhibitory action of inhibitory substances contained in the nucleic acid-containing sample, and thus can be appropriately determined in consideration of the type of polycation, the type of the nucleic acid-containing sample, the pH value of the solution for preparing a sample, a mixing ratio of the nucleic acid-containing sample and the solution for preparing the sample, or the like. For example, in the case of containing polylysine as a polycation, the final concentration of polylysine in the solution for preparing a sample is preferably within a range of 0.01 to 1.0 mw % (% by milli-weight), is more preferably within a range of 0.0125 to 0.8 mw %, is still more preferably within a range of 0.05 to 0.4 mw %. In the present description, the term “mw %” refers to “% by ×10⁻³-weight”.

On the other hand, by the solution for preparing a sample, which is used to mix with a nucleic acid-containing sample such as stool, having a chelating agent, it is enabled to effectively elute and remove inhibitory substances which are contained abundantly in the nucleic acid-containing sample to the solution for preparing a sample. it is carried out previously to add a chelating agent to a preparation solution for a biological sample in order to inhibit reactions by a degrading enzyme and the like, thereby preventing decomposition of biological components such as nucleic acids. But it is a novel finding obtained by inventors of the present invention for the first time that a chelating agent is capable of effectively removing inhibitory substances.

In other words, by conducting the method for preparing a sample with using a solution for preparing a sample containing a chelating agent as an active ingredient, a prepared sample capable of recovering highly purified nucleic acids decreased the carryover of inhibitory substances on nucleic acid analysis. And then, by recovering nucleic acids from the prepared sample, it is able to significantly decrease the carryover of inhibitory substances, thereby recovering highly purified nucleic acids. For this reason, it is able to improve the reliability of nucleic acid analysis by using the recovered nucleic acids.

Examples of the chelating agent used in the present invention include ethylendiaminetetraacetic acid (EDTA), O,O′-bis(2-aminophenyl)ethylene glycol-N,N,N′,N′-tetraacetic acid (BAPTA), N,N-Bis(2-hydroxyethyl)glycine (Bicine), trans-1,2-diaminocyclohexane-ethylendiaminetetraacetic acid (CyTDA), 1,3-diamino-2-hydroxypropane-tetraacetic acid (DPTA-OH), diethylene-triamine-pentaacetic acid (DTPA), ethylendiamine dipropanoic acid hydrochloride, ethylendiamine-2-methylene phosphonic acid hydrate (EDDPO), N-(2-hydroxyethyl)ethylendiamine trisacetic acid (EDTA-OH), ethylendiamine tetra(methylene phosphonic acid) (EDTPO), O,O′-bis(2-aminoethyl)ethylene glycol tetraacetic acid (EGTA), N,N-bis (2-hydroxybenzyl)ethylenediamine diacetic acid (HBED), 1,6-hexamethylenediamine tetraacetic acid (HDTA), N-(2-hydroxyethyl)iminodiacetic acid (HIDA), iminodiacetic acid (IDA), 1,2-diaminopropane tetraacetic acid (Methyl-EDTA), nitrilotriacetic acid (NTA), nitrilotripropanoic acid (NTP), nitrilotris(methylphosphonic acid), trisodium salt (NTPO), N,N,N′,N′-tetrakis(2-pyridylmethyl)ethylenediamine (TPEN), and triethylene tetraamine-hexaacetic acid (TTHA). The solution for preparing of the present invention may contain only a single type of chelating agent or may contain two or more types of chelating agents.

The concentration of chelating agent as a protease inhibitor in the solution for preparing of the present invention is not particularly limited as long as it is a concentration capable of removing inhibitory substances present in the nucleic acid-containing sample, and thus can be appropriately determined in consideration of the types of the nucleic acid-containing sample, the types of chelating agent, or the like. It is preferred that each chelating agent is added so that the final concentration in the solution for preparing a sample is within a range of 0.1 mM to 1 M.

Biological samples such as stool and blood contain large amounts of degrading enzymes and the like as well as inhibitory substances, so nucleic acids therein are easily decomposed by degradation and the like. In particular, stool contains large amounts of digestive remnants and bacteria and nucleic acids therein are decomposed extremely easily. Consequently, in order to improve the accuracy and specificity of nucleic acid analysis, it is important to prevent decomposition of nucleic acids in the steps such as that of preparing a biological sample, that of storing, and that of recovering nucleic acids, and to store the sample stably until the time of the testing procedure.

In addition to a polycation and a chelating agent, the solution for preparing of the present invention preferably contains a water-soluble organic solvent as an active ingredient of the solution. By mixing a nucleic acid-containing sample with the solution having a water-soluble organic solvent as an active ingredient, loss of nucleic acids contained in the nucleic acid-containing sample due to decomposition and the like can be held to a minimum, and nucleic acids can be stored extremely stably, and furthermore, it is able to efficiently recover nucleic acids from the nucleic acid-containing sample. The reason why the water-soluble organic solvent demonstrates such an effect of highly efficient recovery of nucleic acid, in other words, the effect of preventing decomposition of nucleic acids and the like, and storing stably nucleic acids, thereby recovering nucleic acids highly efficiently, is speculated that the dehydrating action possessed by the water-soluble organic solvent would considerably lower cellular activity of organisms having nucleic acids such as indigenous intestinal bacteria, mammalian cells or viruses, thereby changes in nucleic acids inhibited. The reason is also speculated that the protein denaturing action possessed by the water-soluble organic solvent component would considerably decrease the activities of various decomposing enzymes such as protease, DNase or RNase present in the nucleic acid-containing sample such as stool.

Biological samples such as stool usually contain a large amount of water. Therefore, due to using a water-soluble organic solvent, which is a solvent having high water solubility or a solvent capable of mixing at an arbitrary ratio with water, as an active ingredient, the solution for preparing of the present invention is able to rapidly mix with a nucleic acid-containing sample, thereby further increasing the efficiency of nucleic acid recovery.

In the present invention, the term “water-soluble organic solvent” refers to alcohols, ketones, aldehydes, and combinations of these solvents, and these solvents have straight chain structures and are in a liquid state at a temperature close to room temperature, for example, from 15° C. to 40° C. By containing a water-soluble organic solvent with a straight chain structure as an active ingredient, the solution is able to mix with a nucleic acid-containing sample more rapidly than by containing a water-soluble organic solvent with a cyclic structure such as a benzene ring as an active ingredient.

Since organic solvents having a cyclic structure typically easily separate from water, they do not easily mix with biological samples such as stool, and it is difficult for them to obtain superior effects of efficient recovery of nucleic acid. This is because, even in the case of a solvent which is soluble in water to a certain extent, in order to homogeneously disperse biological samples such as stool therein, the sample needs to be mixed vigorously or be heated in many cases. In order to make the mixing of the organic solvents having a cyclic structure with a nucleic acid-containing sample easier, it is also possible to prepare a mixed solution of organic solvents and water in advance, followed by the mixing of a nucleic acid-containing sample with the mixed solution. However, for preparing the mixed solution, the organic solvents having a cyclic structure and water need to be mixed vigorously or be heated in many cases, which is not preferable.

In the solution for preparing of the present invention, the water-soluble organic solvent preferably has a water solubility of 12% by weight or more, more preferably a water solubility of 20% by weight or more, still more preferably a water solubility of 90% by weight or more, and it is most preferable that the water-soluble organic solvent be one which can be mixed with water at a given ratio. Examples of the water-soluble organic solvent which can be mixed with water at a given ratio include methanol, ethanol, n-propanol, 2-propanol, acetone and formaldehyde.

The water-soluble organic solvent contained in the solution for preparing of the present invention is not particularly limited as long as it satisfies the above definition and is a solvent that demonstrates the effect of highly efficient recovery of nucleic acid. Examples of the water-soluble organic solvent include alcohols which are water-soluble alcohols such as methanol, ethanol, propanol, butanol and mercaptoethanol; ketones such as acetone and methyl ethyl ketone (having a water solubility of 90% by weight); aldehydes such as acetaldehyde (acetyl aldehyde), formaldehyde (formalin), glutaraldehyde, paraformaldehyde and glyoxal. Propanol may be either n-propanol or 2-propanol. Further, butanol may be either 1-butanol (having a water solubility of 20% by weight) or 2-butanol (having a water solubility of 12.5% by weight). The water-soluble organic solvent used in the present invention is preferably a water-soluble alcohol, acetone, methyl ethyl ketone or formaldehyde. This is because these solvents have sufficiently high water solubility. From the viewpoints of availability, handling ease, safety and the like, the water-soluble organic solvent is more preferably a water-soluble alcohol, and still more preferably ethanol, propanol or methanol. In particular, ethanol is particularly useful in the screening test for routine health examinations or the like since it is the safest and can easily be handled even in the home.

The concentration of the water-soluble organic solvent in the solution for preparing of the present invention is not particularly limited as long as it is a concentration that demonstrates the effect of highly efficient recovery of nucleic acid, and thus can be appropriately determined in consideration of the types of water-soluble organic solvent or the like. For example, when a water-soluble alcohol or ketone is used as an active ingredient, the concentration of the water-soluble organic solvent in the solution for preparing of the present invention is preferably 30% or more. If the concentration of the water-soluble organic solvent is sufficiently high, when a nucleic acid-containing sample and the solution for preparing a sample are mixed, the water-soluble organic solvent component rapidly penetrates into the whole of nucleic acid-containing sample, thereby enabling the effect of highly efficient recovery of nucleic acid and the effect of decreasing inhibitory action to be demonstrated rapidly.

In particular, when a water-soluble alcohol is used as an active ingredient, the concentration of the water-soluble organic solvent in the solution for preparing of the present invention is preferably 30% or more, more preferably 50% or more, still more preferably within a range of 50 to 80%, and most preferably within a range of 60 to 70%. If the concentration of the water-soluble organic solvent is high, even when the nucleic acid-containing sample has a large amount of water such as stool, the sufficiently effect of highly efficient recovery of nucleic acid can be achieved by adding a small amount of the solution for preparing a sample to the nucleic acid-containing sample.

When acetone or methyl ethyl ketone is used as an active ingredient, the concentration of the water-soluble organic solvent in the solution for preparing of the present invention is preferably 30% or more, more preferably 60% or more, and still more preferably 80% or more. Alternatively, when acetaldehyde, formaldehyde, glutaraldehyde, paraformaldehyde or glyoxal is used as an active ingredient, the concentration of the water-soluble organic solvent in the solution for preparing of the present invention is preferably within a range of 0.01 to 30%, more preferably within a range of 0.03 to 10%, and still more preferably within a range of 3 to 5%. Aldehydes are able to demonstrate the effect of highly efficient recovery of nucleic acid at lower concentrations than alcohols or ketones.

In addition, the water-soluble organic solvent used in the present invention may only contain a single type of water-soluble organic solvent or may be a mixed solution of two or more types of water-soluble organic solvents. For example, the water-soluble organic solvent may be a mixed solution of two or more types of alcohols, or may be a mixed solution of an alcohol and another type of water-soluble organic solvent. A mixed solution of alcohol and acetone is preferable since nucleic acid recovery efficiency is further improved.

The pH of the solution for preparing of the present invention is preferably acidic. This is to more effectively inhibit hydrolysis of nucleic acids. The pH of the solution for preparing of the present invention is preferably within a range of 2 to 6.5, more preferably within a range of 3 to 6, and still more preferably within a range of 4.5 to 5.5.

The solution for preparing of the present invention preferably has buffering action so that the pH thereof fluctuates a little and is maintained within the aforementioned pH ranges after adding a certain amount of acid or base, particularly a biological sample such as a stool, to the solution. As the solution for preparing a sample having a buffering action, a solution prepared by adding the active ingredient such as polycation, a chelatin agent, or a water-soluble organic solvent to an appropriate buffer solution may be used. In this invention, the solution for preparing a sample preferably contains an organic acid and a conjugate base of that organic acid and demonstrates buffering action attributable to the organic acid and the conjugate base thereof. For example, the solution for preparing a sample may be adjusted to a desired pH by adding an organic acid and an alkaline metal salt or alkaline earth metal salt of that organic acid. The pH thereof may also be adjusted by using a hydroxide of an alkaline metal or alkaline earth metal after adding the organic acid.

In addition, the solution for preparing of the present invention may be a solution that contains both organic acid and mineral acid and has suitable buffering action. For example, the solution for preparing a sample may be a solution obtained by mixing a water-soluble organic solvent with a buffer system having buffering action in the acidic range such as a glycine/HCl buffer system, sodium cacodylate/HCl buffer system or potassium hydrogen phthalate/HCl buffer system.

In the present invention, the pH of the solution for preparing a sample is the value obtained by measuring with a pH meter of which the measuring principle is the glass electrode method (such as that manufactured by DKK-Toa Corp.) after having calibrated with a phthalate standard solution and neutral phosphate standard solution.

In addition, the solution for preparing of the present invention may contain any components other than the polycations, chelating agents, and water-soluble organic solvents, provided they do not impair the effect of decreasing inhibitory action achieved by polycation or chelating agent, and provided they do not impair the effect of highly efficient recovery of nucleic acid achieved by water-soluble organic solvent in the case when the solution contain a water-soluble organic solvent as an active ingredient. For example, the solution for preparing a sample may contain a chaotropic salt or a surface active agent. The containing of a chaotropic salt or a surface active agent makes it possible to more effectively inhibit cellular activity and enzyme activity of various decomposing enzymes present in nucleic acid-containing samples. Examples of chaotropic salts that can be added to the solution for preparing a sample include guanidine hydrochloride, guanidine isothiocyanate, sodium iodide, sodium perchlorate and sodium trichloroacctatc. A nonionic surface active agent is preferable for the surface active agent able to be added to the solution for preparing a sample. Examples of these nonionic surface active agents include Tween 80, CHAPS (3-[3-cholamidopropyl-dimethylammonio]-1-propane sulfonate), Triton X-100 and Tween 20. The type and concentration of chaotropic salt or surface active agent are not particularly limited as long as it is a component with a concentration that allows the obtaining of the effect of decreasing inhibitory action achieved by polycation or chelating agent, and of the effect of highly efficient recovery of nucleic acid achieved by water-soluble organic solvent, and can be appropriately determined in consideration of the amount of the nucleic acid-containing sample, the methods for recovering and analyzing a nucleic acid employed afterwards, or the like.

In addition, a colorant may be added to the solution for preparing a sample, where appropriate. By coloring the solution for preparing a sample, various effects can be achieved, such as the prevention of accidental swallowing and the lightening of the sample in the case of using stool as a nucleic acid-containing sample. The colorant is preferably a coloring agent used as a food additive, and is preferably blue, green, or the like. Examples of colorants include Fast Green FCF (Green No. 3), Brilliant Blue FCF (Blue No. 1) and indigo carmine (Blue No. 2). Further, a plurality of colorants may be added as a mixture, or a single colorant may be added.

In the method for preparing a sample according to the present invention, mixing a nucleic acid-containing sample with the solution for preparing of the present invention may be conducted by immersing the nucleic acid-containing sample in the solution for preparing a sample without a particular stirring treatment. The solution for preparing of the present invention is very easy to mix with biological samples having a large amount of water such as stool, so if the amount and condition of the nucleic acid-containing sample are suitable, the solution for preparing a sample penetrates sufficiently into the nucleic acid-containing sample and obtains the effect of decreasing inhibitory action or the effect of highly efficient recovery of nucleic acid even when the nucleic acid-containing sample is just immersed in the solution for preparing a sample without particularly stirring.

To mix a nucleic acid-containing sample with the solution for preparing of the present invention may be conducted by putting and immersing the nucleic acid-containing sample in the solution for preparing a sample and followed by stirring. Stirring make it possible to more sufficiently disperse and suspend the nucleic acid-containing sample in the solution for preparing a sample. In the case of putting the nucleic acid-containing sample into the solution for preparing a sample and stirring to mix, it is preferred that the mixing is carried out conducted promptly. By dispersing the nucleic acid-containing sample in the solution for preparing a sample promptly, the solution for preparing a sample is able to rapidly penetrate into cells or nucleic acids present in the nucleic acid-containing sample, and the effect of decreasing inhibitory action or the effect of highly efficient recovery of nucleic acid can be obtained quickly.

Furthermore, the method used to mix the nucleic acid-containing sample and the solution for preparing a sample is not particularly limited as long as it is a method involving physical operations. For example, the mixing may be carried out by putting the nucleic acid-containing sample in a container in which the solution for preparing a sample has been contained in advance, followed by immersing the nucleic acid-containing sample in the solution for preparing a sample during storage. In addition, it may be carried out by putting the nucleic acid-containing sample such as a collected stool in a sealable container in which the solution for preparing a sample has been contained in advance, followed by vertically inverting the container or shaking the container using a shaker, such as a vortex mixer. Furthermore, the nucleic acid-containing sample and the solution for preparing a sample may be mixed under the presence of particles for mixing.

A method that uses a shaker or a method that uses particles for mixing is preferable for this mixing method since the mixing can be carried out rapidly. In particular, in the case of using stool as a nucleic acid-containing sample, by using a stool collection container in which particles for mixing are contained in advance, the mixing of the stool and the solution for preparing a sample can be rapidly conducted even in environments with no special equipment such as the home.

The particles for mixing are not particularly limited as long as they are formed of compositions that do not impair the effect of decreasing inhibitory action achieved by polycation or chelating agent and the effect of highly efficient recovery of nucleic acid achieved by the water-soluble organic solvent component, and are particles having hardness and specific gravity sufficient to rapidly disperse a nucleic acid-containing sample in the solution for preparing a sample by colliding with the stool. The particles may be composed of one type of material or may be composed of two or more types of materials. Examples of such particles for mixing include particles composed of glass, ceramics, plastics, latex, metals, or the like. In addition, the particles for mixing may be magnetic particles or nonmagnetic particles.

The volume of the solution for preparing a sample to be mixed with the nucleic acid-containing sample is not particularly limited. In terms of the mixing ratio of the nucleic acid-containing sample and the solution for preparing a sample, the volume of the solution for preparing a sample is preferably 1 or more, relative to 1 volume of the nucleic acid-containing sample. If the volume of the solution for preparing a sample contained in a container is equivalent to or more than the volume of the nucleic acid-containing sample, when the nucleic acid-containing sample is a sample in solid form or in partially solid form such as stool, the whole of the nucleic acid-containing sample can be completely immersed in the solution, and thus the effects of the present invention can be achieved more effectively. For example, in the case when the volume of the nucleic acid-containing sample and that of the solution for preparing a sample are equivalent, it becomes possible to reduce the weight and size of the container that contains the solution for preparing a sample. On the other hand, by mixing a nucleic acid-containing sample with the solution for preparing a sample whose volume is five times or more than that of the nucleic acid-containing sample, the nucleic acid-containing sample can be effectively and rapidly dispersed in the solution, and the adverse effects caused by the decline of water-soluble alcohol concentration due to the water contained in the nucleic acid-containing sample can also be suppressed. Since a proper balance can be achieved between the two effects; i.e., the weight reduction of a container that contains the solution for preparing a sample, and the improvement of dispersibility of the nucleic acid-containing sample, the mixing ratio of the nucleic acid-containing sample and the solution for preparing a sample is preferably within a range of 1:1 to 1:20, more preferably within a range of 1:3 to 1:10 and still more preferably about 1:5.

The amount of the nucleic acid-containing sample supplied for the method for preparing a sample of the present invention is not particularly limited, but is preferably within a range of 10 mg to 1 g. If the amount of the nucleic acid-containing sample is too large, the collection procedure requires more effort and the size of a container in which the nucleic acid-containing sample is collected and prepared also becomes too large, thereby resulting in deterioration of the handling property or the like. On the other hand, in the case when the amount of the nucleic acid-containing sample is too small, the number of cells contained in the nucleic acid-containing sample is too small, and the necessary amount of nucleic acid cannot be recovered, thereby resulting in reducing the level of analytical accuracy for the target nucleic acid. In addition, in the case when stool is used as a nucleic acid-containing sample, since stool is heterogeneous, in other words, various kinds of components are non-uniformly present therein, the stool sample is preferably collected from various parts of the stool in order to avoid the adverse effects caused by the localization of mammalian cells.

The effect of highly efficient recovery of nucleic acid achieved by the water-soluble organic solvent as described above is not particularly subjected to the effects of temperature conditions provided an adequate amount of the water-soluble organic solvent is present. Thus, the method for preparing a sample of the present invention enables nucleic acids derived from the nucleic acid-containing sample to be stably stored with holding the loss of nucleic acids in a prepared sample even in the case of carrying out the method at a temperature at which a nucleic acid-containing sample is generally collected, namely even in the case of carrying out the method at room temperature. In addition, the prepared sample is able to stably preserve nucleic acids in the sample even in the case of storing or transporting at room temperature. However, the storage temperature is preferably 50° C. or lower. The reason for this is that there is the risk of the concentration of the water-soluble organic solvent in the prepared sample decreasing below the concentration sufficient for demonstrating the effect of highly efficient recovery of nucleic acid by volatilization and the like as a result of storing for a long period of time under a temperature of 50° C. or higher.

The prepared sample prepared by using the method for preparing a sample according to the present invention may be recovered nucleic acids immediately after the mixing the nucleic acid-containing sample and the solution for preparing a sample. It is preferably that the prepared sample is stored for a predetermined amount of time prior to be supplied to the step of nucleic acid recovery. It is because that, in the case when a chelating agent is used as an active ingredient of the solution for preparing a sample, enough amounts of inhibitory substances can be removed by storing for a predetermined amount of time. In the case when a polycation is used as an active ingredient of the solution for preparing a sample, the storage for a predetermined amount of time allows polycations to sufficiently interact with inhibitory substances deprived from the nucleic acid-containing sample, thereby decreasing the inhibitory action effectively.

Note that, if the amount of the nucleic acid-containing sample is small or the condition of the nucleic acid-containing sample is suitable, it is able to recover highly purified nucleic acids from which inhibitory substances are sufficiently removed or which inhibitory actions are sufficiently decreased, even in the case of carrying out the nucleic acid recovery immediately after mixing the nucleic acid-containing sample with the solution for preparing a sample.

In the case of storing for a predetermined amount of time after mixing the nucleic acid-containing sample with the solution for preparing a sample, the duration of storage of the prepared sample is not particularly limited provided it is an amount of time that allows the obtaining of the effect of the present invention, and thus can be appropriately determined in consideration of the type of the nucleic acid-containing sample, the type and concentration of the polycation and/or the chelating agent, the type and concentration of the water-soluble organic solvent, the mixing ratio of the nucleic acid-containing sample and the solution for preparing a sample, the storage temperature or the like. In the method for preparing a sample according to the present invention, the storage time of the prepared sample (that is, the mixture of the nucleic acid-containing sample and the solution for preparing a sample) is preferably 1 hour or more, more preferably 12 hours or more, still more preferably 24 hours or more and particularly preferably 72 hours or more. In addition, the storage time may also be 168 hours or more. For example, by storing the prepared sample prepared by mixing a stool and the solution for preparing of the present invention (to also be referred to as the “stool sample of the present invention”) for at least 1 hour after the mixing, it is able to remove inhibitory substances to the extent that effects of carryover of inhibitory substances can be inhibited under the general reaction condition of PCR.

The prepared sample of the present invention enables to effectively improve the preservation efficiency of nucleic acids present in the nucleic acid-containing sample, particularly nucleic acids which are only present in comparatively small amounts in the nucleic acid-containing sample such as nucleic acids derived from mammalian cells and to sufficiently decrease inhibitor actions of inhibitory substances, due to decreasing actions against inhibitory actions achieved by the polycation or the chelating agent, or due to the dehydrating actions, protein denaturing actions and nucleic acid decomposition inhibitory actions achieved by the water-soluble organic solvent. Thus, when a nucleic acid-containing sample is prepared by the method for preparing a sample according to the present invention, highly reliable analysis results can be expected to be obtained by using the prepared sample having been stored for a long period or having been transported, as well as by using the prepared sample immediately after preparation.

Furthermore, it is general that in the case of the target nucleic acid being present in a trace amount, nucleic analysis using an enzyme reaction where nucleic acid is used as substrate is easily subjected to the effects of inhibitory substances, rather than in the case of the target nucleic acid being present in a comparatively large amount. On the other hand, the recovery from the prepared sample of the present invention allows inhibitory actions of inhibitory substances to be significantly decreased, and the reliability of the nucleic analysis can be improved by using the recovered nucleic acids.

In the case of using the solution for preparing a sample further having a water-soluble organic solvent as an active ingredient thereof, in particular, the method for preparing a sample of the present invention is preferably used for preparing a sample which is used for recovering a nucleic acid from a stool in order to analyze nucleic acids present in stool. It is because the effect of recovering highly purified nucleic acids in an efficient manner (the effect of high efficiency of recovering purified nucleic acid recovery) can be obtained from the method even in the case of using stool which contains large amounts of contaminants such as inhibitory substances and nucleic acid-degrading enzymes. The reason why the significantly excellent effect of high efficiency of recovering purified nucleic acid recovery can be obtained by the method is not apparent, but it is speculated as indicated below:

Since stool has large amounts of inhibitory substances rather than the other biological sample such as blood, it is often difficult to remove an adequate amount of inhibitory substances in a simple and easy manner. For example, in the case of the washing with a solution having an action of removing inhibitory substances or the like, the amount of removed inhibitory substances by a one-time washing operation is inadequate in many cases. It is thought that more inhibitory substances can be removed by repeating the washing operation several times or by making the period of time of the each washing operation. However, the stool sample contains various kinds of components derived from stool, and the risk of the loss of nucleic acids by decomposition and the like becomes higher as the period of time of the washing operation is longer. In addition, it is unsuitable for routine health examinations and the like which require the preparation of a lot of stool samples to carry out a washing operation for several times, as the amount of waste solution becomes large.

On the other hand, the stool sample prepared by mixing a stool with the solution for preparing of the present invention further having a water-soluble organic solvent as an active ingredient thereof allows nucleic acids to be stably stored even at room temperature for a long period of time, mainly due to the action of a water-soluble organic solvent. Consequently, the prepared stool sample allows nucleic acids to be stably stored for a sufficient period which it needs to remove an adequate amount of inhibitory substances, even at room temperature, thereby recovering a sufficient amount of nucleic acids. In other words, by having both an active ingredient achieving the effect of decreasing inhibitory actions, such as the chelating agent and polycation, and the water-soluble organic solvent as active ingredient of the solution for preparing a sample, it is able to prepare a stool sample from which highly purified nucleic acids can be recovered highly efficiently.

As described above, the stool sample prepared by mixing a stool with the solution for preparing of the present invention effectively improves the preservation efficiency of nucleic acids present in stool, particularly nucleic acids which are only present in comparatively small amounts in stool such as nucleic acids derived from mammalian cells and to remove an adequate amount of inhibitory substances, due to decreasing actions against inhibitory actions achieved by the polycation or the chelating agent, or due to the dehydrating actions, protein denaturing actions and nucleic acid decomposition inhibitory actions achieved by the water-soluble organic solvent. Thus, when a stool is prepared by the method for preparing a sample according to the present invention, highly reliable analysis results can be expected to be obtained by using the stool sample having been stored for a long period or having been transported, as well as by using the stool sample immediately after preparation.

### <A Method for Analyzing a Nucleic Acid Contained in a Nucleic Acid-Containing Sample>

The prepared sample of the present invention can be applied to various nucleic acid analyses in the same manner as other biological samples containing nucleic acids. It is particularly preferably used in nucleic acid analyses for investigating the development of cancer or infectious diseases for which early detection is important. In addition, it is preferably used in nucleic acid analyses for investigating for the development of inflammatory diseases such as colitis, enteritis, gastritis or pancreatitis. It may also be used for testing for protruding lesions such as polyps as well as testing for diseases of the large intestine, small intestine, stomach, liver, gallbladder and bile duct, such as gastric ulcer.

More specifically, the method for analyzing a nucleic acid contained in a nucleic acid-containing sample is conducted as the following. First, a nucleic acid-containing sample is mixed with the solution for preparing of the present invention to prepare a sample, and a nucleic acid is recovered from cells contained in the prepared sample, followed by analyzing the recovered nucleic acid. The method for recovering a nucleic acid from a cell contained in the prepared sample and the following method for analyzing are not particularly limited, and can be appropriately adopted from conventionally known methods for recovery and analysis.

<A Method for Preparing a Sample in the Case when a Nucleic Acid-Containing Sample is a Stool>

In the case when a nucleic acid-containing sample is a stool, by preparing a stool sample from a collected stool using the method for preparing a sample of the present invention, a nucleic acid present in stool, and particularly a nucleic acid derived from a mammalian cell, can be stably preserved at room temperature for an extended period of time while minimizing changes over time in molecular profiling of mammalian cells such as the cells exfoliated from the large intestine contained in the stool. Consequently, even in cases in which time is required from stool collection to nucleic acid analysis or in cases in which the location where the stool sample is collected is a considerable distance away from the location where nucleic acids are analyzed, such as screening examinations including routine health examinations, the stool sample prepared by a method for preparing a sample of the present invention can be stored or transported while inhibiting decomposition of nucleic acids, and particularly decomposition of fragile RNA. In addition, special equipment for refrigerating or freezing and the setting of storage temperature conditions are not required and stool samples can be stored or transported easily and at low cost.

In particular, when analyzing, as the target nucleic acid, a nucleic acid derived from an organism other than indigenous intestinal bacterium, in other words, the nucleic acid contained in a stool in a relatively small amount as compared to the nucleic acid derived from indigenous intestinal bacterium which are contained therein in a large amount, it is preferable to prepare a stool sample using the solution for preparing of the present invention. Nucleic acids in stool are gradually lost over time following the stool excretion due to degradation or the like. For this reason, when the target nucleic acids are those that are present in stool in a small amount, if an analysis is performed using a stool sample in which the degradation of nucleic acids has already taken place, it may not be possible to recover a sufficient amount of target nucleic acids for the analysis. Accordingly, it is highly probable that the results would appear negative (i.e., the target nucleic acids are absent in the stool), even if the target nucleic acids were present in the stool immediately after the stool excretion. By preparing a stool sample using the solution for preparing of the present invention, the nucleic acids in the stool can be stably preserved, as a result of which the nucleic acids in the stool can be sufficiently recovered even if they are present therein in a small amount, thereby improving the accuracy and sensitivity of nucleic acid analysis.

Examples of the above-mentioned nucleic acids derived from an organism other than indigenous intestinal bacterium include nucleic acids derived from mammalian cells, such as nucleic acids derived from cancer cells, and nucleic acids from causative microorganisms responsible for infectious diseases in the early stage or late stage of those infectious diseases, such as hepatitis viruses. In addition, the nucleic acids may be derived from parasites.

Note that in the present invention, the term “indigenous intestinal bacterium/bacteria” refers to the bacterial cells which are relatively abundant in stool and are usually living inside the intestines of animals such as humans. Examples of such indigenous intestinal bacteria include obligate anaerobes such as those belonging to the genera of Bacteroides, Eubacterium, Bifidobacterium and Clostridium; and facultative anaerobes such as those belonging to the genera of Escherichia, Enterobacter, Klebsiella, Citrobacter and Enterococcus

It is possible to examine the development of cancers, such as colon cancer and pancreatic cancer, for example, by detecting and analyzing the nucleic acids derived from cancer cells, in other words, the nucleic acids that are carrying mutations and the like, from the stool sample. In addition, by examining whether the nucleic acids derived from causative microorganisms responsible for the infectious diseases, such as the nucleic acids derived from viruses or the nucleic acids derived from parasites, can be detected or not from the stool sample, it is possible to examine the development of infectious diseases or the presence and absence of parasites. In particular, by using the stool sample for the detection of causative microorganisms excreted in the stool, such as hepatitis A and E viruses, a test for infectious diseases can be carried out in a noninvasive, simple and easy manner. In addition, by examining whether the nucleic acids derived from pathogenic bacteria other than indigenous intestinal bacteria, for example, bacteria causing food poisoning such as enterohemorrhagic Escherichia coli 0-157 strain, can be detected or not, development of microbisms can also be tested.

It is particularly preferable to detect a marker indicating neoplastic transformation or a marker indicating an inflammatory gastrointestinal disease. Examples of the marker indicating neoplastic transformation include conventionally known cancer markers, such as carcinoembryonic antigen (CEA) and sialyl Tn antigen (STN), and the presence and absence of mutations in the APC gene, p53 gene, K-ras gene, or the like. Further, detection of methylation of genes, such as p16, hMLHI, MGMT, p14, APC, E-cadherin, ESR1 and SFRP2, is also useful as a diagnostic marker for colon diseases (for example, refer to Lind et al., “A CpG island hypermethylation profile of primary colorectal carcinomas and colon cancer cell lines” Molecular Cancer, 2004, Vol. 3, No. 28). In addition, it has already been reported that the DNA derived from Helicobacter pylori in a stool sample may be used as a marker for gastric cancer (for example, refer to Nilsson et al., Journal of Clinical Microbiology, 2004, Vol. 42, No. 8, pp. 3781-3788). Meanwhile, the Cox-2 gene or the like, for example, is known as a marker indicating inflammatory gastrointestinal disease. Cox gene is also used as a marker indicating neoplastic transformation.

Various kinds of materials are present in the stool sample, and a large number of substances which may become inhibiting factors in the nucleic acid analyses are also present therein. For this reason, it is possible to further improve the analytical accuracy by first recovering the nucleic acids from the stool sample and then performing the nucleic acid analyses using the recovered nucleic acids. As mentioned above, since nucleic acids can be recovered highly efficiently from the stool sample prepared by the method for preparing a sample of the present invention and the inhibitory action of inhibitory substances deprived from stool can be efficiently decreased in the stool sample, the sample is highly suitable, not only for the analysis of nucleic acids derived from indigenous intestinal bacteria which are present in the stool in large numbers, but also for the analysis of nucleic acids derived from mammalian cells which are present in a small amount. Since the sample is formed of stool, it is preferably used for the analysis of nucleic acids derived from cells of gastrointestinal tracts, such as the large intestine, small intestine and stomach, and it is particularly preferable that the nucleic acids derived from cells exfoliated from the large intestine be analyzed using the sample.

The method for recovering nucleic acids from stool samples is not particularly limited, and any type of method may be adopted as long as it is a method generally used when recovering nucleic acids from samples. The stool sample of the present invention contains mainly nucleic acids derived from an organism other than indigenous intestinal bacterium, such as mammalian cells (hereafter, may be referred to as “mammalian cells or the like”), and nucleic acids derived from indigenous intestinal bacterium. In the nucleic acid recovery from stool samples, although nucleic acids derived from mammalian cells or the like and nucleic acids derived from indigenous intestinal bacteria may be recovered separately, it is particularly preferable to recover them simultaneously. Simultaneously recovering nucleic acids derived from mammalian cells or the like and nucleic acids derived from indigenous intestinal bacteria allows nucleic acids derived from indigenous intestinal bacteria which are highly abundant in stool to function as carriers. As a result, nucleic acids derived from mammalian cells or the like which are present in small numbers can be recovered much more efficiently, as compared to the cases where the nucleic acids are recovered following the isolation of mammalian cells or the like from the stool. Note that nucleic acids recovered from stool samples may be DNA, RNA, or a mixture of DNA and RNA.

For example, nucleic acids derived from mammalian cells or the like and nucleic acids derived from indigenous intestinal bacteria can be recovered simultaneously from the stool sample of the present invention by performing, as a step (a), denaturing of a protein in the stool sample of the present invention, thereby extracting nucleic acids from mammalian cells or the like and indigenous intestinal bacteria in the stool sample; and then, as a step (b), recovery of the extracted nucleic acids.

The denaturing of proteins in the stool sample in the step (a) can be carried out using a conventionally known technique. For example, by adding a compound generally used as a denaturing agent of proteins, such as a chaotropic salt, an organic solvent or a surface active agent, to the stool sample, proteins in the stool sample can be denatured. As the chaotropic salt or surface active agent to be added to the stool sample in the step (a), the same chaotropic salts and surface active agents as those mentioned earlier to be added to the solution for preparing of the present invention can be used. Phenol is preferable as the above organic solvent. Phenol may be neutral or acidic. When acidic phenol is used, it is possible to selectively extract RNA rather than DNA in an aqueous layer. When adding a chaotropic salt, an organic solvent, a surface active agent or the like to the stool sample in the step (a), one type of compound may be added, or two or more types of compounds may be added.

Following the step (a) and prior to the step (b), as a step (c), the protein denatured in the step (a) may be removed. By removing the denatured proteins before recovering nucleic acids, it is possible to improve the quality of recovered nucleic acids. The removal of proteins in the step (c) can be carried out using a conventionally known technique. For example, denatured proteins can be removed by precipitating the denatured proteins by centrifugation, followed by the collection of supernatant alone. Rather than simply performing a centrifugal separation process, denatured proteins can even more thoroughly removed by first adding chloroform to a sample, and subsequently stirring and mixing the resultant sufficiently using a vortex mixer or the like, and the denatured proteins are then precipitated by centrifugation, followed by the collection of supernatant alone.

The recovery of the extracted nucleic acids in the step (b) can be carried out by a known technique such as an ethanol precipitation method and a cesium chloride ultracentrifugation method. Moreover, nucleic acids can be recovered by first, as a step (b1), making the nucleic acids extracted in the step (a) to adsorb to an inorganic substrate; and then, as a step (b2), eluting the nucleic acids adsorbed in the step (b1) from the inorganic substrate.

As the inorganic substrate to which nucleic acids are adsorbed in the step (b-1), a conventionally known inorganic substrate which is capable of adsorbing nucleic acids can be used. In addition, the shape of the inorganic substrate is not particularly limited, and it may be a particulate form or a membranous form. Examples of the inorganic substrate include silica-containing particles (beads) such as silica gel, siliceous oxide, glass and diatomaceous earth; and porous membranes made of nylon, polycarbonate, polyacrylate, and nitrocellulose.

As a solvent for eluting the adsorbed nucleic acids in the step (b2) from the inorganic substrate, a solvent generally used for eluting nucleic acids from conventionally known inorganic substrates can be used, where appropriate, determined in consideration of the type of recovered nucleic acids or the method for the following nucleic acid analysis. Purified water is particularly preferable as the solvent for elution. Furthermore, it is preferable to wash the inorganic substrate to which nucleic acids are adsorbed with an appropriate washing buffer, following the step (b1) and prior to the step (b2).

In the case a stool sample is prepared using a solution for preparing a sample which contains a chaotropic salt or a surface active agent at a concentration sufficient for extracting nucleic acids from mammalian cells or the like, the step (a) can be omitted in the recovery of nucleic acids from the stool sample.

When a stool sample is prepared using a solution for preparing a sample which does not contain a chaotropic salt or a surface active agent at a concentration sufficient for eluting nucleic acids from mammalian cells or the like, as a step (d), it is preferable to recover a solid component from the stool sample prior to the step (a). In order to rapidly mix the stool with the solution for preparing a sample, the stool sample contains a larger proportion of liquid components with respect to the solid components, which are derived from the stool. Accordingly, by removing the liquid components from the stool sample and then recovering only the solid components containing mammalian cells or the like and indigenous intestinal bacteria, it is possible to reduce the scale of the samples used for recovering and analyzing nucleic acids. Moreover, by removing a water-soluble organic solvent from the solid components, it is also possible to suppress the adverse effects of the water-soluble organic solvent in the step for recovering nucleic acids from the solid components. For example, by centrifuging the stool sample of the present invention to precipitate the solid components therein and then removing the supernatant, the solid components alone can be recovered. Alternatively, it is also possible to recover the solid components alone by a filtration process or the like. Further, it is also preferable to wash the recovered solid components with an adequate buffer such as phosphate buffered saline (PBS, pH 7.4).

Furthermore, although a denaturing agent of proteins, such as a chaotropic salt, may be added directly to the recovered solid components, it is preferable to first suspend the solid components in an adequate medium and then add a denaturing agent of proteins thereto. When recovering DNA, as an extraction agent, for example, a phosphate buffer, a tris buffer, or the like can be used. It is preferable that DNases in the extraction agent be deactivated by high pressure steam sterilization or the like, and it is more preferable that the extraction agent contains a protease such as Proteinase K. On the other hand, when recovering RNA, as the extraction agent, for example, a citrate buffer or the like can be used. However, since RNA is a material which is highly prone to degradation, it is preferable to use a buffer containing an RNase inhibitor, such as guanidine thiocyanate and guanidine hydrochloride.

Depending on the analytical methods used afterwards, the recovery of nucleic acids from the stool sample may not be needed. More specifically, after extracting nucleic acids from mammalian cells or the like and indigenous intestinal bacteria in the stool sample, the sample can be directly used for the nucleic acid analysis. For example, when pathogenic bacteria and the like are present in large numbers in a stool sample and if the nucleic acids from the pathogenic bacteria were to be analyzed, it is possible to detect genes or the like derived from pathogenic bacteria by first recovering a solid components from the stool sample and then adding thereto an extraction agent, such as PBS, which contains a protease, such as Proteinase K, to mix, and finally using the obtained uniform solution of stool sample directly for the nucleic acid analysis. Alternatively, the recovery of nucleic acids from the stool sample can also be carried out by using a commercially available kit such as a nucleic acid extraction kit or a virus detection kit.

The nucleic acids recovered from the stool sample of the present invention can be analyzed using a conventionally known analytical method. Examples of the method for analyzing nucleic acids include a method for quantitating nucleic acids and a method for detecting specific base sequence regions using polymerase chain reaction (PCR) or the like. In addition, when RNA is recovered, it is possible to first synthesize cDNA by reverse transcriptase reaction, and then analyze the synthesized cDNA in the same manner as described above for the DNA analysis. For example, by detecting the presence or absence of a base sequence region containing a cancer gene or the like or a base sequence region containing microsatellites, it is possible to examine the development of cancers. When using the DNA recovered from the stool sample, for example, the analysis of mutations in the DNA or the analysis of epigenetic changes can be performed. Examples of the mutation analysis include the analyses of insertion, deletion, substitution, duplication and inversion of one or more bases. Examples of the analysis of epigenetic changes include the analyses of methylation and demethylation. On the other hand, when using the recovered RNA, for example, it is possible to detect mutations in the RNA, such as the insertion, deletion, substitution, duplication and inversion of one or more bases, and splicing variants (isoforms). In addition, the analyses of functional RNA (non-coding RNA), such as the analyses of, for example, transfer RNA (tRNA), ribosomal RNA (rRNA) and microRNA (miRNA), can be carried out. Furthermore, the level of RNA expression can also be detected and analyzed. It is particularly preferable to perform an mRNA expression analysis, a mutation analysis of K-ras gene, an analysis of DNA methylation, or the like. These analyses can be carried out according to the methods which are conventionally known in this field. Moreover, it is also possible to use a commercially available analysis kits such as a K-ras gene mutation analysis kit and a methylation detection kit.

In this manner, nucleic acids present in stool can be analyzed with high sensitivity and high accuracy by using the method for preparing a sample according to the present invention, the method for recovering nucleic acids from a stool sample prepared according to this preparation method, and a nucleic acid analysis method that uses nucleic acids recovered according to this nucleic acid recovery method. Consequently, this can be expected to contribute and be applicable to early detection and diagnosis of various symptoms and diseases, including colon cancer, observation of the course of treatment, and pathological research on other abnormal states and the like.

By collecting stool in a stool collection container in which the solution for preparing of the present invention is contained in advance, a collected stool can be prepared in an even more simple and rapid manner. In addition, by using a kit for collecting stool that includes both the solution for preparing of the present invention and a stool collection container containing the solution for preparing a sample, the effects of the present invention can be achieved more easily. Note that the kit for collecting stool may include a constituent other than the solution for preparing a sample and the stool collection container containing the solution, such as a stool collection rod, where appropriate.

The form or size of such stool collection container is not particularly limited, and known stool collection containers which may be able to contain a solvent can be used. A stool collection container in which the lid of the stool collection container and a stool collection rod are integrated into a single unit is preferable because it is easy to handle. In addition, because the amount of stool collected can be controlled, the stool collection rod which is able to collect a predetermined fixed amount of stool is more preferable. Examples of such stool a collection container which is already known include a stool collection container disclosed in Japanese Examined Patent Application, Second Publication No. H6-72837.

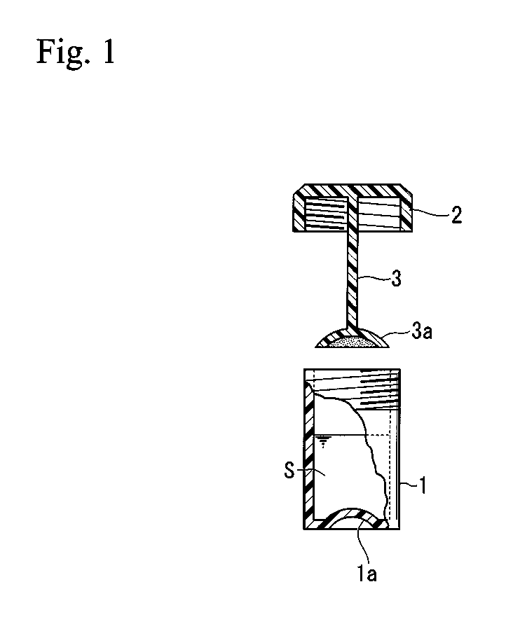

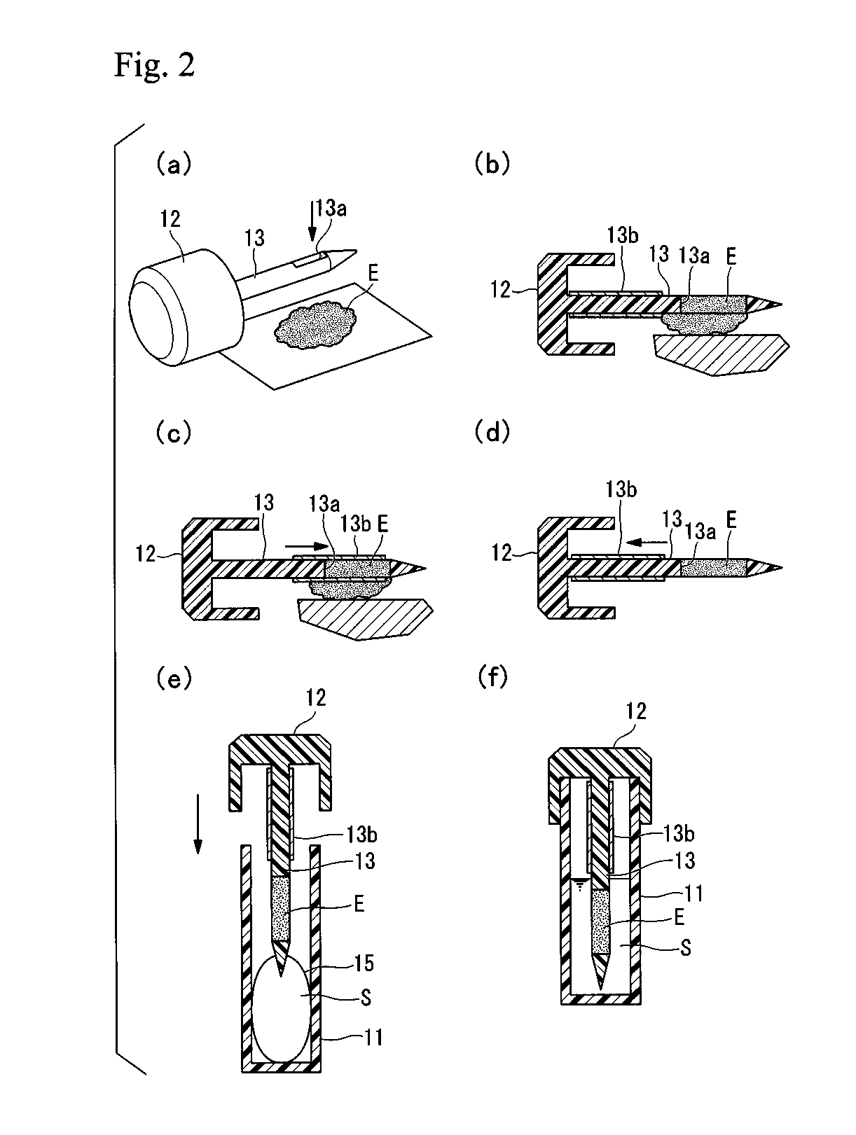

FIGS. 1 and 2 are diagrams showing one aspect of a stool collection container which can be used for a kit for collecting stool according to the present invention. It should be noted that the stool collection containers which can be used for a kit for collecting stool according to the present invention are not limited to these stool collection containers.

First, a stool collection container in FIG. 1 will be described. The stool collection container includes a lid 2 which is integrated with a stool collection rod 3, and a container body 1, and contains the solution S for preparing a sample according to the present invention therein. A cup 3a which may collect a predetermined amount of stool is attached to the top end of the stool collection rod 3, and the cup 3a has sieve mesh. Meanwhile, a protruded portion 1a having a shape which is complementary to that of the cup 3a is present in the bottom of the container body 1. By fitting the cup 3a with the protruded portion 1a, the stool collected in the cup 3a is mechanically extruded from the sieve mesh in the cup 3a, and thus the stool can be rapidly dispersed in the solution S for preparing a sample.

The stool collection container depicted in FIG. 2 is a stool collection container that includes a lid 12 integrated with a stool collection rod 13 having a pointed end; a container body 11; and a bag 15, which is sealed and contains the solution S for preparing a sample according to the present invention, inside the container body 11. An orifice 13a for collecting a certain amount of stool E is formed in the stool collection rod 13. In addition, a movable lid 13b which may become a lid for the orifice 13a by sliding over the stool collection rod 13 is also attached. As shown in FIG. 2a, the movable lid 13b is first slid to the lid 12 side across the orifice 13a so as to leave the orifice 13a in a completely open state, and then the stool collection rod 13 is pressed against the stool E. Then, as shown in FIG. 2b, the orifice 13a is filled with the stool E. In this state, the movable lid 13b is slid to cover the orifice 13a, thereby accurately collecting in an amount equal to the volume of the slot 13a (FIG. 2c). Thereafter, the movable lid 13b is returned to the original position so as to make the orifice 13a in a completely open state (FIG. 2d), and then the lid 12 is housed in the container body 11 (FIG. 2e). When the stool collection rod 13 is housed in the container body 11, because the pointed end of the stool collection rod 13 breaks the bag 15 containing the solution S for preparing a sample, the solution S for preparing a stool sample and the stool E are mixed. Since such a stool collection container is filled with a solution only after the stool collection rod is placed inside the container, even when using a solution for preparing a sample which is harmful for the human body, such as methanol, accidents due to the solution leakage can be avoided, and thus the container can be handled safely even in the home.

<A Method for Analyzing a Nucleic Acid Recovered from a Nucleic Acid-Containing Sample>

The effect of decreasing inhibitory actions achieved by polycation is able to obtain by adding polycation to reaction solutions for nucleic acid analysis using reverse transcription reaction or nucleic acid chain elongation reaction. In other words, the method for a analyzing a nucleic acid according to another aspect of the present invention is characterized in that a nucleic acid recovered from a nucleic acid-containing sample is analyzed by a reverse transcription reaction or a nucleic acid chain elongation reaction in a reaction solution containing polycations. Examples of the nucleic acid chain elongation reaction include the same nucleic acid chain elongation reaction as those mentioned earlier.

As a nucleic acid supplied to the method for a analyzing a nucleic acid according to another aspect of the present invention, a nucleic acid recovered from a prepared sample prepared by the method for preparing a sample of the present invention may be used, and a nucleic acid recovered from a nucleic acid-containing sample having been prepared by a method other than the method for preparing a sample of the present invention my also used.

The same polycation as those mentioned earlier can be used for the polycation added to the reaction solution. In the method for analyzing a nucleic acid according to another aspect of the present invention, the polycation is preferably polylysine or polyacrylamide, and more preferably polylysine. The reaction solution may contain only a single type of polycation or may contain two or more types of polycations.

The composition of the reaction solution used for the reverse transcription reaction or the nucleic acid chain elongation reaction may be a typical one except polycation added, and reagents generally used for reverse transcription reaction or nucleic acid chain elongation reaction can be used at concentrations generally used. The reaction condition for the reverse transcription reaction or the nucleic acid chain elongation reaction may be determined in consideration of the type of enzyme, the Tm values of primers, or the like, and the reaction may be carried out by a method generally used. The ways to determine the reaction condition are well known in the present technical field.

Preferred embodiments of the present invention are explained above, but the present invention is not limited to these embodiments. Additions, omissions, replacement, and other modifications in the constitution can be made without departing from the spirit or scope of the present invention. Other than this, the invention is not restricted by the above description, but only by the scope of the appended claims.

Next, the present invention will be described in more detail based on a series of examples, although the scope of the present invention is in no way limited by the following examples. Note that Caco-2 cells, which were cultured cells, were cultured by ordinary methods. In addition, polylysines used in the examples were manufactured by Sigma-Aldrich Corporation.

### Example 1

Stool collected from one healthy individual was dispensed into eight 15-mL polypropylene tubes (0.5 g each). To each stool, 10 mL of a 60% ethanol solution (pH5.5, Stool Sample 1-1), a polylysine (the final concentration: 0.0125 mw %)-containing 60% ethanol solution (pH5.5, Stool Sample 1-2), a polylysine (the final concentration: 0.025 mw %)-containing 60% ethanol solution (pH5.5, Stool Sample 1-3), a polylysine (the final concentration: 0.05 mw %)-containing 60% ethanol solution (pH5.5, Stool Sample 1-4), a polylysine (the final concentration: 0.1 mw %)-containing 60% ethanol solution (pH5.5, Stool Sample 1-5), a polylysine (the final concentration: 0.2 mw %)-containing 60% ethanol solution (pH5.5, Stool Sample 1-6), a polylysine (the final concentration: 0.4 mw %)-containing 60% ethanol solution (pH5.5, Stool Sample 1-7), or a polylysine (the final concentration: 0.8 mw %)-containing 60% ethanol solution (pH5.5, Stool Sample 1-8), was added, as the solution for preparing a sample, and dispersed well to prepare Stool Sample 1-1 to 1-8. The final pH values of all the solutions for preparing a sample that were used for preparation of Stool Sample 1-1 to 1-8 were adjusted to 5.5 with a 0.1 M citric acid/sodium hydroxide solution.

After storing these stool samples for one day at 25° C., RNA was recovered from each stool sample. More specifically, the solid components of the stool were recovered by centrifuging each tube. Then, a phenol mixture “Trizol” (manufactured by Invitrogen Corporation) was added to the obtained solid components, and the samples were sufficiently mixed using a homogenizer, followed by the addition of chloroform. After sufficiently mixing the resultant by using a vortex mixer, the mixtures were centrifuged (12,000×g) at 4° C. for 20 minutes. The supernatant (aqueous layer) obtained as a result of the centrifugation was passed through an RNA recovery column of the RNeasy Midi Kit (manufactured by Qiagen GmbH). RNA was recovered by carrying out a washing procedure and RNA elution procedure on the RNA recovery column of this kit in accordance with the protocol provided.

RT-PCR was carried out on 1 μg of the recovered RNA to detect the human GAPDH (Glyceraldehyde-3-phosphate dehydrogenase) gene from the recovered DNA. PCR was carried out using the resulting cDNA as a template followed by detection of human GAPDH gene. The GAPDH primer probe MIX (manufactured by Applied Biosystems, Inc., Catalog No.: Hs02786624_gl) was used as primer.

More specifically, 1 μL of the recovered DNA was first dispensed into each well of 96-well PCR plate. Subsequently, 8 μL of ultra-pure water and 10 μL of the nucleic acid amplification reagent “TaqMan Gene Expression Master Mix” (manufactured by Applied Biosystems, Inc.) were added to each well, 1 μL of GAPDH Primer Probe MIX (manufactured by Applied Biosystems, Inc.) were each added thereto and mixed, thereby preparing PCR reaction solutions.

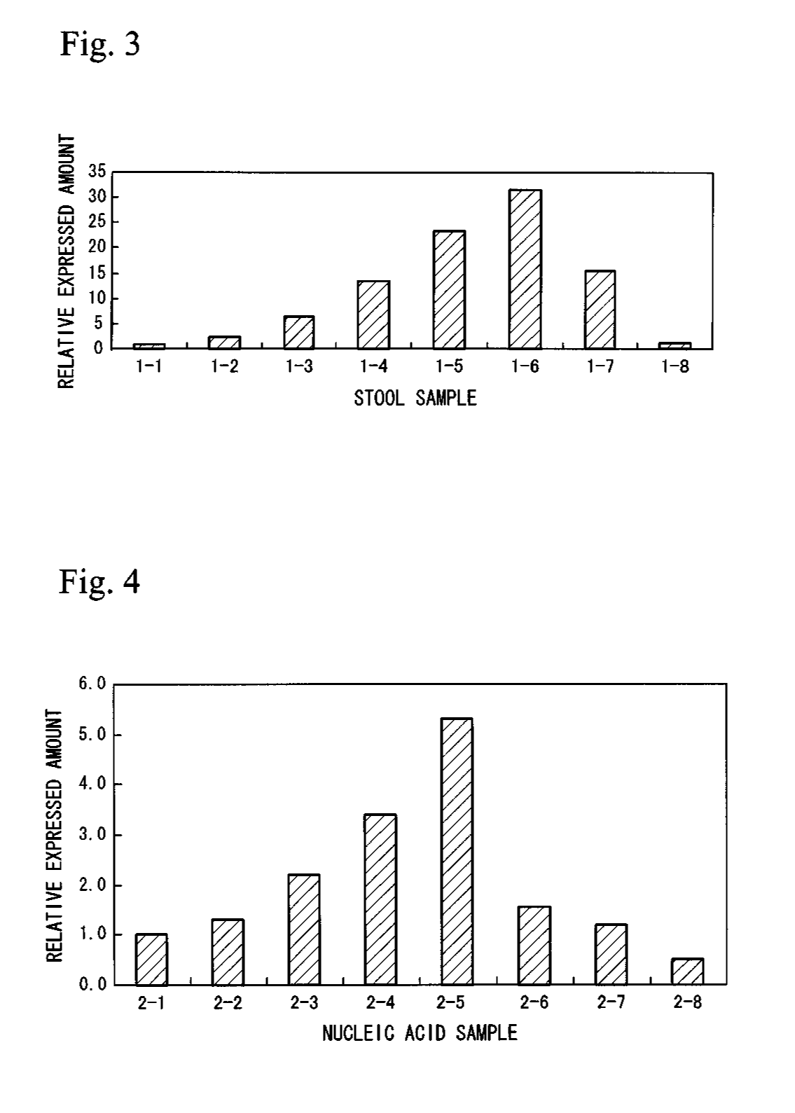

PCR was carried out while measuring fluorescence intensity over time by placing this PCR plate in an ART real-time PCR apparatus, and initially treating for 10 minutes at 95° C. followed by carrying out 40 cycles of heat cycling consisting of 1 minute at 95° C., 1 minute at 56.5° C. and 1 minute at 72° C., and then further treating for 7 minutes at 72° C. By analyzing the results of fluorescence intensity measurements, the relative values of the expressed amount of GAPDH gene in the RNA recovered from each sample were calculated. The results of a relative comparison of the expressed amounts of GAPDH gene in RNA derived from each stool sample are shown in FIG. 3.

As shown in FIG. 3, in the case of using nucleic acids recovered from the stool samples which were prepared by using solutions containing both polycation (polylysine) and water-soluble organic solvent (ethanol), the amount of the target nucleic acid (human GAPDH gene) detected by PCR was larger and the reaction efficiency was higher than in the case of using nucleic acids recovered from the stool sample which was prepared by using a solution containing only water-soluble organic solvent. Furthermore, from the fact that nucleic acids recovered from Stool Sample 1-6 had the highest reaction efficiency, it was confirmed that the action of decreasing inhibitory actions of inhibitory substances achieved by polylysine may have an optimum concentration and the reaction is decreased inversely by high concentrations.

From the above results, it is evident that by using the solution for preparing a sample containing a polycation and a water-soluble organic solvent, a stool sample be prepared from which nucleic acids decreased inhibitory actions of inhibitory substances deprived stool are recovered with high efficiency.

### Example 2

A nucleic acid elongation reaction was carried out in a polycation-containing reaction solution.

More specifically, first, stool collected from one healthy individual was added directly by a phenol mixture “Trizol” (manufactured by Invitrogen Corporation) and sufficiently mixed using a homogenizer, followed by the addition of chloroform. After sufficiently mixing the resultant by using a vortex mixer, the mixtures were centrifuged (12,000×g) at 4° C. for 20 minutes. The supernatant (aqueous layer) obtained as a result of the centrifugation was passed through an RNA recovery column of the RNeasy Midi Kit (manufactured by Qiagen GmbH). RNA was recovered by carrying out a washing procedure and RNA elution procedure on the RNA recovery column of this kit in accordance with the protocol provided.

RT (reverse transcriptase reaction) was carried out on 1 μg of the recovered RNA in a polycation-containing reaction solution. The cDNA obtained form an RT reaction measurements, the relative values of the expressed amount of GAPDH gene in the nucleic solution without polylysine was named Nucleic Acid Sample 2-1, and the cDNAs obtained from RT reaction solutions containing polylysine at the final concentration of 0.125 mw %, 0.25 mw %, 0.5 mw %, 1 mw %, 2 mw %, 4 mw %, or 8 mw % were named Nucleic Acid Samples 2-2 to 2-8, respectively.

PCR was carried out on 1 μg of these nucleic acid samples (cDNA) to detect the human GAPDH (Glyceraldehyde-3-phosphate dehydrogenase) gene. PCR was carried out in the same manner as Example 1, using the GAPDH primer probe MIX (Catalog No.: Hs02786624_gl) as primer. By analyzing the results of fluorescence intensity acid samples were calculated. The results of the calculation are shown in FIG. 4.

As a result, it was confirmed that in spite of all of the RT reaction solutions containing the equivalent amounts of RNA, the expressed amounts of GAPDH gene of Nucleic Acid Samples 2-2 to 2-7, in which RT were carried out in reaction solutions containing polylysine at final concentration of 0.125 to 4 mw %, were higher than that of Nucleic Acid Sample 2-1, in which RT was carried out in a reaction solution without polylysine. The reason is thought to be that the reaction efficiencies of Nucleic Acid Sample 2-2 to 2-7 were higher than that of Nucleic Acid Sample 2-1. It was also confirmed that the reaction efficiency was lower in Nucleic Acid Sample 2-8 than in Nucleic Acid Sample 2-1, and that, as well as in Example 1, the high concentration of polylysine cause the reaction inhibition.

From the above results, it is evident that by using the solution for preparing a sample containing a polycation and a water-soluble organic solvent, it is able to decrease inhibitory action by inhibitory substances carried over in the reaction solution with nucleic acids, thereby improving the reaction efficiency.

### Example 3

RNA was recovered from stool collected from one healthy individual in the same manner as Example 2.

RT was carried out on 1 μg of the recovered RNA in a polycation, calcium chloride (manufactured by Wako Pure Chemical Industries, Ltd.), or magnesium chloride hexahydrate (manufactured by Wako Pure Chemical Industries, Ltd.)-containing reaction solution. The cDNA obtained from an RT reaction solution without polylysine, calcium chloride, or magnesium chloride hexahydrate was named Nucleic Acid Sample 3-1. The cDNA obtained from an RT reaction solution containing polylysine at a final concentration of 1 mw % was named Nucleic Acid Sample 3-2. The cDNA obtained from RT reaction solutions containing calcium chloride at a final concentration of 0.1 mM, 1 mM, or 10 mM were named Nucleic Acid Samples 3-3 to 3-5 respectively, and the cDNA obtained from RT reaction solutions containing magnesium chloride hexahydrate at a final concentration of 0.1 mM, 1 mM, or 10 mM were named Nucleic Acid Samples 3-6 to 3-8 respectively.

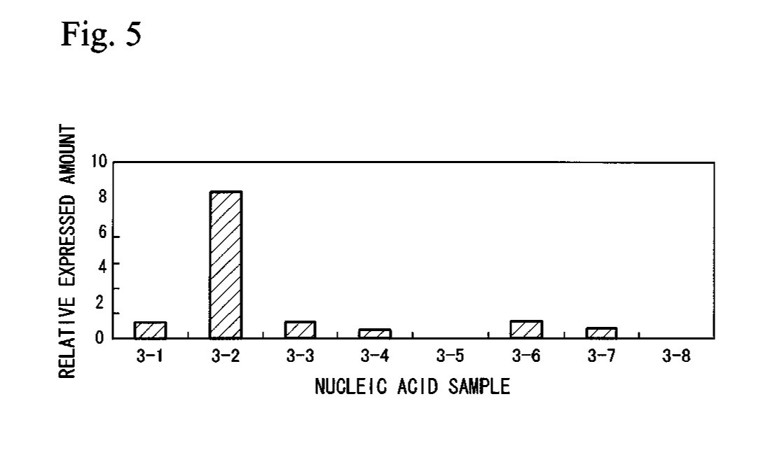

PCR was carried out on 1 μg of these nucleic acid samples (cDNA) to detect the human GAPDH (Glyceraldehyde-3-phosphate dehydrogenase) gene. PCR was carried out in the same manner as Example 1, using the GAPDH primer probe MIX (manufactured by Applied Biosystems, Inc., Catalog No.: Hs02786624_gl) as primer. By analyzing the results of fluorescence intensity measurements, the relative values of the expressed amount of GAPDH gene in the nucleic acid samples were calculated. The results of the calculation are shown in FIG. 5.

As a result, it was confirmed that the expression level of GAPDH gene of Nucleic Acid Sample 3-2, in which RT was carried out in a reaction solution containing polylysine, was eight times higher than that of Nucleic Acid Sample 3-1 as a control sample. On the contrary, the reaction efficiencies of Nucleic Acid Sample 3-4 to 3-8, in which RT were carried out in a reaction solution containing calcium chloride, or magnesium chloride hexahydrate, were decreased in a concentration-dependent manner. In detail, in nucleic acid samples containing calcium chloride, the expressed amount of Nucleic Acid Sample 3-3 at the final concentration of 0.1 mM was nearly equal to that of Nucleic Acid Sample 3-1, but, that of Nucleic Acid Sample 3-4 at the final concentration of 1 mM was about half of that of Nucleic Acid Sample 3-1, and that of Nucleic Acid Sample 3-5 at the final concentration of 10 mM was below the detection limit. Similarly, in nucleic acid samples containing magnesium chloride hexahydrate, the expressed amount of Nucleic Acid Sample 3-6 at the final concentration of 0.1 mM was nearly equal to that of Nucleic Acid Sample 3-1, but, that of Nucleic Acid Sample 3-7 at the final concentration of 1 mM was about half of that of Nucleic Acid Sample 3-1, and that of Nucleic Acid Sample 3-8 at the final concentration of 10 mM was below the detection limit.

From these results, it was confirmed that containing polylysine in a reaction solution enables the reaction efficiency of RT-PCR to improve, but the reaction is inhibited by adding calcium chloride or magnesium chloride hexahydrate to a reaction solution. It is reported that a high concentration of metal ions inhibits PCR-reaction (see, for example, Applied and Environmental Microbiology, 1998, Vol. 64, No. 10, pp. 3748-3753), so it is speculated that PCR reactions were inhibited by metal ion brought in the PCR reaction solutions from the RT reaction solutions. For this reason, in the case of containing high concentration of metal ion in an RT reaction solution, prior to being used for PCR, the obtained cDNA is required to be purified by an ethanol precipitation method and the like. On the contrary, since a polylysine does not inhibit a PCR reaction, even when a polylysine is added to an RT reaction solution, the obtained cDNA can be used directly for PCR without being purified, and has an excellent effect of decreasing inhibitory actions.

### Example 4

In the same manner as Example 2, a nucleic acid elongation reaction was carried out in a polycation-containing reaction solution.

More specifically, first, stool collected from one colorectal cancer patient who was prospectively confirmed the expression of Cox-2 gene, which is a marker indicating a neoplastic transformation, was added directly by a phenol mixture “Trizol” (manufactured by Invitrogen Corporation) and sufficiently mixed using a homogenizer, followed by the addition of chloroform. After sufficiently mixing the resultant by using a vortex mixer, the mixtures were centrifuged (12,000×g) at 4° C. for 20 minutes. The supernatant (aqueous layer) obtained as a result of the centrifugation was passed through an RNA recovery column of the RNeasy Midi Kit (manufactured by Qiagen GmbH). RNA was recovered by carrying out a washing procedure and RNA elution procedure on the RNA recovery column of this kit in accordance with the protocol provided.

RT (reverse transcriptase reaction) was carried out on 1 μg of the recovered RNA in a polycation-containing reaction solution. The cDNA obtained form an RT reaction measurements, the relative values of the expressed amount of GAPDH gene in the nucleic solution without polylysine was named Nucleic Acid Sample 4-1, and the cDNAs obtained from RT reaction solutions containing polylysine at the final concentration of 0.125 mw %, 0.25 mw %, 0.5 mw %, 1 mw %, 2 mw %, 4 mw %, or 8 mw % were named Nucleic Acid Samples 4-2 to 4-8, respectively.

PCR was carried out on 1 μg of these nucleic acid samples (cDNA) to detect the human Cox-2 gene. PCR was carried out in the same manner as Example 1, using the Cox-2 primer probe MIX (manufactured by Applied Biosystems, Inc.) as primer. By analyzing the results of fluorescence intensity acid samples were calculated.

As a result, as well as in Example 2, it was confirmed that in spite of all of the RT reaction solutions containing the equivalent amounts of RNA, the expressed amounts of Cox-2 gene of Nucleic Acid Samples 4-2 to 4-7, in which RT were carried out in reaction solutions containing polylysine at final concentration of 0.125 to 4 mw %, were higher than that of Nucleic Acid Sample 4-1, in which RT was carried out in a reaction solution without polylysine. The reason is thought that the reaction efficiencies of Nucleic Acid Samples 4-2 to 4-7 were higher than that of Nucleic Acid Sample 4-1. It was also confirmed that the reaction efficiency was lower in Nucleic Acid Sample 4-8 than in Nucleic Acid Sample 4-1, and that, as well as in Example 1, the high concentration of polylysine cause the reaction inhibition.

### Example 5

Stool collected from one healthy individual was dispensed into five 15-mL polypropylene tubes (0.5 g each). To each stool, 10 mL of a 60% ethanol solution (pH5.5, Stool Sample 5-1), an EDTA (the final concentration: 0.1 mM)-containing 60% ethanol solution (pH5.5, Stool Sample 5-2), an EDTA (the final concentration: 1 mM)-containing 60% ethanol solution (pH5.5, Stool Sample 5-3), or an EDTA (the final concentration: 10 mM)-containing 60% ethanol solution (pH5.5, Stool Sample 5-4), was added, as the solution for preparing a sample, and dispersed well to prepare stool samples. The final pH values of all the solutions for preparing a sample that were used for preparation of Stool Sample 5-1 to 5-4 were adjusted to 5.5 with a 0.1 M citric acid/sodium hydroxide solution.

After storing these stool samples for 7 days at 25° C., RNA was recovered from each stool sample. More specifically, the solid components of the stool were recovered by centrifuging each tube. Then, a phenol mixture “Trizol” (manufactured by Invitrogen Corporation) was added to the obtained solid components, and the samples were sufficiently mixed using a homogenizer, followed by the addition of chloroform. After sufficiently mixing the resultant by using a vortex mixer, the mixtures were centrifuged (12,000×g) at 4° C. for 20 minutes. The supernatant (aqueous layer) obtained as a result of the centrifugation was passed through an RNA recovery column of the RNeasy Midi Kit (manufactured by Qiagen GmbH). RNA was recovered by carrying out a washing procedure and RNA elution procedure on the RNA recovery column of this kit in accordance with the protocol provided. All of the amounts of RNA recovered from Stool Samples 5-1 to 5-4 were about 40 μg, the RNA yields were nearly equal amounts of each other.

RT-PCR was carried out on 1 μg of the recovered RNA to detect the human GAPDH gene from the recovered DNA. The GAPDH primer probe MIX (manufactured by Applied Biosystems, Inc., Catalog No.: Hs02786624_gl) was used as primer.

More specifically, 1 μg of the recovered DNA was first dispensed into each well of 96-well PCR plate. Subsequently, 8 μL of ultra-pure water and 10 μL of the nucleic acid amplification reagent “TaqMan Gene Expression Master Mix” (manufactured by Applied Biosystems, Inc.) were added to each well, 1 μL of GAPDH Primer Probe MIX (manufactured by Applied Biosystems, Inc.) were each added thereto and mixed, thereby preparing PCR reaction solutions.

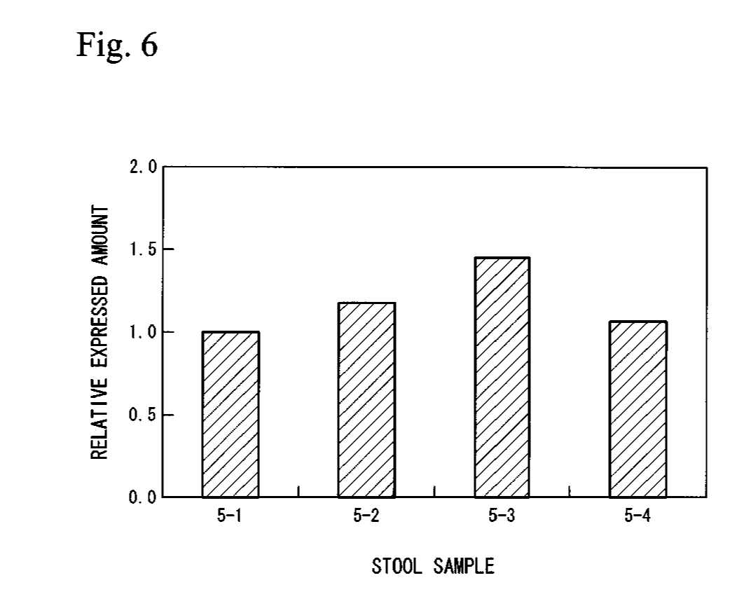

PCR was carried out while measuring fluorescence intensity over time by placing this PCR plate in an ABI real-time PCR apparatus, and initially treating for 10 minutes at 95° C. followed by carrying out 40 cycles of heat cycling consisting of 1 minute at 95° C., 1 minute at 56.5° C. and 1 minute at 72° C., and then further treating for 7 minutes at 72° C. By analyzing the results of fluorescence intensity measurements, the relative values of the expressed amount of GAPDH gene in the RNA recovered from each sample were calculated. The results of a relative comparison of the expressed amounts of GAPDH gene in RNA derived from each stool sample are shown in FIG. 6.

As shown in FIG. 6, in the case of using nucleic acids recovered from the stool samples which were prepared by using solutions containing both chelating agent (EDTA) and water-soluble organic solvent (ethanol), the amount of the target nucleic acid (human GAPDH gene) detected by PCR was larger and the reaction efficiency was higher than in the case of using nucleic acids recovered from the stool sample which was prepared by using a solution containing only water-soluble organic solvent.

Although, the amounts of the recovered RNA from these tool samples were nearly equal to each other independently of whether a chelating agent has been added, nucleic acids recovered from stool samples which have been prepared by the solution for a sample containing a chelating agent had higher reaction efficiency of PCR than those recovered from stool samples which have been prepared by the solution for a sample not containing a chelating agent. It is because that the RNA recovered form a stool sample with a chelating agent has less carryover of inhibitory substances on PCR derived from stool and higher purity than one recovered form a stool sample without a chelating agent. Consequently, from these results, it is evident that by using the solution for preparing a sample containing a chelating agent and a water-soluble organic solvent, a stool sample can be prepared from which highly purified nucleic acids which are recovered with high efficiency.

### Example 6

A nucleic acid elongation reaction was carried out in an EDTA-containing reaction solution.

More specifically, first, stool collected from one colorectal cancer patient who was prospectively confirmed the expression of Cox-2 gene, which is a marker indicating a neoplastic transformation, was dispensed into five 15-mL polypropylene tubes (0.5 g each) in the same manner as Example 1.

To each stool, 10 mL of a 60% ethanol solution (pH5.5, Stool Sample 6-1), an EDTA (the final concentration: 0.1 mM)-containing 60% ethanol solution (pH5.5, Stool Sample 6-2), an EDTA (the final concentration: 1 mM)-containing 60% ethanol solution (pH5.5, Stool Sample 6-3), or an EDTA (the final concentration: 10 mM)-containing 60% ethanol solution (pH5.5, Stool Sample 6-4), was added, as the solution for preparing a sample, and dispersed well to prepare stool samples The final pH values of all the solutions for preparing a sample that were used for preparation of Stool Sample 6-1 to 6-4 were adjusted to 5.5 with a 0.1 M citric acid/sodium hydroxide solution.

After storing these stool samples for 7 days at 25° C., RNA was recovered from each stool sample. More specifically, the solid components of the stool were recovered by centrifuging each tube. Then, a phenol mixture “Trizol” (manufactured by Invitrogen Corporation) was added to the obtained solid components, and the samples were sufficiently mixed using a homogenizer, followed by the addition of chloroform. After sufficiently mixing the resultant by using a vortex mixer, the mixtures were centrifuged (12,000×g) at 4° C. for 20 minutes. The supernatant (aqueous layer) obtained as a result of the centrifugation was passed through an RNA recovery column of the RNeasy Midi Kit (manufactured by Qiagen GmbH). RNA was recovered by carrying out a washing procedure and an RNA elution procedure on the RNA recovery column of this kit in accordance with the protocol provided. All of the amounts of RNA recovered from Stool Sample 6-1 to 6-4 were about 40 μg, the RNA yields were nearly equal amounts of each other.

RT-PCR was carried out on 1 μg of the recovered RNA to detect the human Cox-2 gene from the recovered DNA. The Cox-2 primer probe MIX (manufactured by Applied Biosystems, Inc.) was used as primer.

More specifically, 1 μL of the recovered DNA was first dispensed into each well of 96-well PCR plate. Subsequently, 8 μL of ultra-pure water and 10 μL of the nucleic acid amplification reagent “TaqMan Gene Expression Master Mix” (manufactured by Applied Biosystems, Inc.) were added to each well, 1 μL of Cox-2 Primer Probe MIX (manufactured by Applied Biosystems, Inc.) were each added thereto and mixed, thereby preparing PCR reaction solutions.

PCR was carried out while measuring fluorescence intensity over time by placing this PCR plate in an ABI real-time PCR apparatus, and initially treating for 10 minutes at 95° C. followed by carrying out 40 cycles of heat cycling consisting of 1 minute at 95° C., 1 minute at 56.5° C. and 1 minute at 72° C., and then further treating for 7 minutes at 72° C. By analyzing the results of fluorescence intensity measurements, the relative values of the expressed amount of Cox-2 gene in the RNA recovered from each sample were calculated.

As well as in Example 5, in the case of using nucleic acids recovered from the stool samples which were prepared by using solutions containing both chelating agent (EDTA) and water-soluble organic solvent (ethanol), the amount of the target nucleic acid (human Cox-2 gene) detected by PCR was larger and the reaction efficiency was higher than in the case of using nucleic acids recovered from the stool sample which was prepared by using a solution containing only water-soluble organic solvent.

Although, the amounts of the recovered RNA from these stool samples were nearly equal to each other independently of whether a chelating agent was added, nucleic acids recovered from stool samples being prepared by the solution for a sample containing a chelating agent had higher reaction efficiency of PCR than those recovered from stool samples which have prepared by the solution for a sample not containing a chelating agent. It is because the RNA recovered form a stool sample with a chelating agent has less carryover of inhibitory substances on PCR derived from stool and higher purity than one recovered form a stool sample without a chelating agent. Consequently, from these results, it is evident that by using the solution for preparing a sample containing a chelating agent and a water-soluble organic solvent, a stool sample can be prepared from highly purified nucleic acids which are recovered with high efficiency.

### Reference Example 1

Stool collected from one healthy individual was dispensed into three 15-mL polypropylene tubes (1.0 g each). Immediately after the dispensation, one polypropylene tube was quickly subjected to a freezing treatment using liquid nitrogen, thereby preparing a stool sample (1A). After the dispensation, 10 mL of 70% ethanol solution was added to one of the other polypropylene tubes. After sufficiently dispersing the stool in the solution, the tube was left statically for 1 hour, thereby preparing a stool sample (1B). After the dispensation, the remaining one polypropylene tube was quickly transferred to an extraction step without adding any solutions or the like thereto, thereby preparing a stool sample (1C).

Thereafter, RNA was recovered from each stool sample. More specifically, 3 mL of a phenol mixture “Trizol” (manufactured by Invitrogen Corporation) was added to each stool sample, and the samples were sufficiently mixed for 30 seconds or more using a homogenizer, followed by the addition of 3 mL of chloroform to each stool sample. After sufficiently mixing the resultant by using a vortex mixer, the mixtures were centrifuged (12,000×g) at 4° C. for 20 minutes. The supernatant (aqueous layer) obtained as a result of the centrifugation was passed through an RNA recovery column of the RNeasy midi kit (manufactured by Qiagen GmbH), and RNA was recovered by the washing of the RNA recovery column followed by RNA extraction according to the protocol provided in the kit. The recovered RNA was quantified using the Nanoprop instrument (manufactured by Nanoprop Technologies, Inc.).

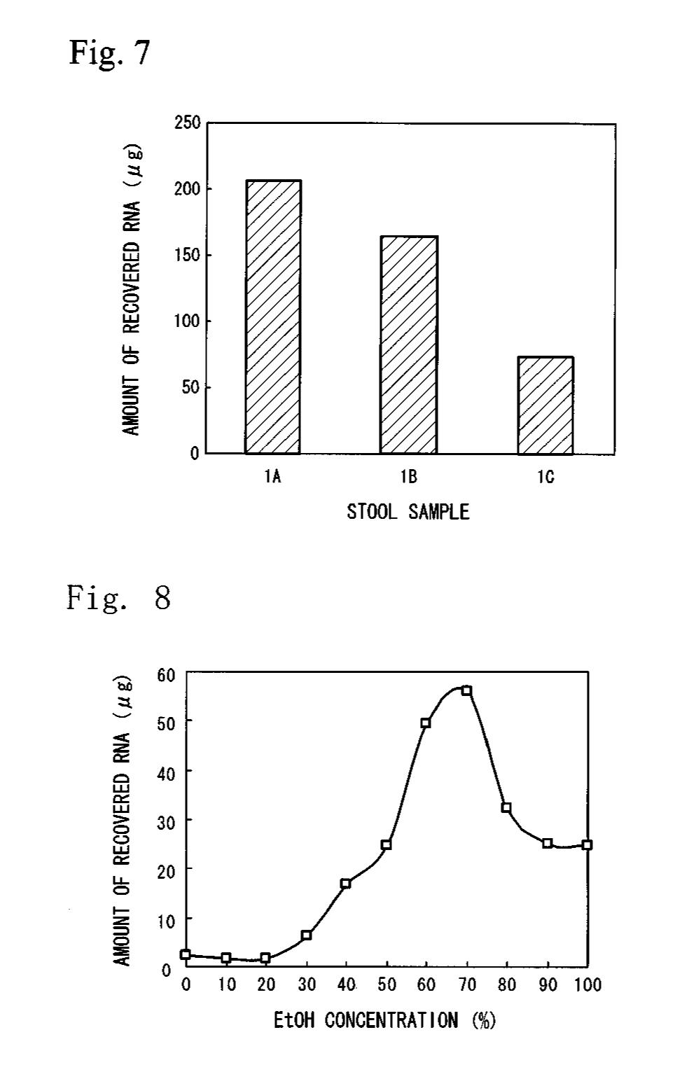

FIG. 7 is a diagram showing the amount of RNA recovered from each stool sample. From the stool sample (1B) prepared using an ethanol solution which was the solution of the present invention for preparing a stool sample, it was possible to recover a much larger amount of RNA, as compared to the stool sample (1C) in which nucleic acids were quickly extracted immediately after the stool collection, although it was slightly less than the amount of RNA recovered from the stool sample (1A) which was subjected to a freezing treatment immediately after the stool collection. From these results, it is evident that even when a preparation process is conducted at room temperature, by using the solution for preparing a sample according to the present invention in the preparation process, it is possible to obtain a stool sample from which nucleic acids may be recovered highly efficiently. In those cases where a patient is collecting stool at home for a checkup or the like, it is desirable that the preparation of stool samples can be carried out at a temperature close to room temperature. The solution for preparing a sample according to the present invention fully satisfies such a requirement.

### Reference Example 2

0.5 g of stool from one healthy individual was mixed with 5.0×10⁵ cells of a human colon cancer cell line (Caco-2 cells) which were expressing a high level of MDR1 (multidrug resistance 1) gene to prepare an artificial stool of colon cancer patients, and this artificial stool was used to prepare stool samples by the method for preparing a stool sample according to the present invention.

More specifically, the artificial stool of colon cancer patients was dispensed into 15-mL polypropylene tubes (0.5 g each), and the solutions for preparing a stool sample indicated in Table 1 were added to each tube and mixed, thereby preparing the stool samples. Note that the “universal collection medium” in the table refers to a preservation medium disclosed in Japanese Translation of PCT Application No. 2004-500897 which contains 500 mL of Puck's Saline G, 400 mg of sodium bicarbonate, 10 g of bovine serum albumin (BSA), 500 units/L of penicillin G, 500 mg/L of streptomycin sulfate, 1.25 mg/L of amphotcricin B and 50 mg/L of gentamicin. The prepared stool samples were preserved in a constant temperature incubator set at room temperature (25° C.) for 1, 3, 7, and 10 days, respectively.

TABLE 1

| Solution for preparing sample | |
|---|---|
| (2A) | 5 mL of 70% methanol solution |
| (2B) | 1 mL of 100% methanol solution |
| (2C) | 5 mL of universal collection medium |
| (2D) | 5 mL of PBS |

Following preservation, RNA was recovered from each stool sample, and attempts were made in order to detect the transcription products (mRNA) of MDR1 gene from the recovered RNA. With respect to the stool sample prepared using the solution for preparing a sample (2C) (hereafter, referred to as the “stool sample (2C)”), mammalian cells including Caco-2 cells were first separated, followed by the RNA recovery. With respect to the stool samples prepared using the solutions for preparing a stool sample other than the solution for preparing a sample (2C), the nucleic acids originating from mammalian cells and the nucleic acids originating from bacteria were recovered at the same time without the separation of mammalian cells. The separation of mammalian cells from the stool sample (2C) was specifically conducted as follows. 5 mL of Histopack 1077 solution (manufactured by Sigma-Aldrich Corporation) was added to the stool sample (2C) and mixed, and the mixture was then centrifuged (200×g) at room temperature for 30 minutes, followed by the recovery of the interfacial portion between the suspension and the Histopack 1077 solution. The separated mammalian cells were washed three times with PBS.

The recovery of RNA from the stool samples was specifically conducted as follows. 3 mL of a phenol mixture “Trizol” (manufactured by Invitrogen Corporation) was first added to the stool sample (or to the separated mammalian cells, only for the case of the stool sample (2C)), and the samples were sufficiently mixed for 30 seconds or more using a homogenizer, followed by the addition of 3 mL of chloroform. Then, the resultant was centrifuged at 12,000×g for 10 minutes. The supernatant (aqueous layer) obtained as a result of the centrifugation was collected in a new polypropylene tube. Thereafter, RNA was recovered from the collected supernatant using the RNeasy midi kit (manufactured by Qiagen GmbH).

Reverse transcriptase polymerase chain reaction (RT-PCR) was performed using the recovered RNA, and PCR was then carried out using the obtained cDNA as a template. As primers, a base sequence for amplifying MDR1 gene which had a sequence number 1 and a base sequence for amplifying MDR1 gene which had a sequence number 2 were used as a forward primer and a reverse primer, respectively.

More specifically, to a 0.2-mL PCR tube, 12 μL of ultra-pure water and 2 μL of a buffer (10×) were added, and 1 μL of cDNA, the forward primer, the reverse primer, magnesium chloride, dNTP, and DNA polymerase were each added thereto and mixed, thereby preparing a PCR reaction solution. PCR was carried out for 30 cycles, each amplification cycle consisted of incubating the PCR tubes at 95° C. for 30 seconds, 60° C. for 30 seconds, and then at 72° C. for 1 minute. The PCR products obtained as a result of the amplification was electrophoresed using the Agilent DNA 1000 LabChip (registered trade mark) kit (manufactured by Agilent Technologies, Inc.), and the intensity of the obtained band was measured, thereby examining the extent of amplification indicated by the PCR products.

TABLE 2

| Preservation periods | 1 day | 3 days | 7 days | 10 days |
|---|---|---|---|---|
| Stool sample (2A) | ++ | ++ | ++ | + |
| Stool sample (2B) | ++ | ++ | + | + |
| Stool sample (2C) | − | − | − | − |
| Stool sample (2D) | − | − | − | − |

++: Intense level of amplification; +: Intermediate level of amplification; +/−: Weak level of amplification; −: No amplification

Table 2 summarizes the extent of amplification indicated by the PCR products which originated from each stool samples, based on different preservation periods. Note that in the table, “stool sample (2A)” refers to a stool sample prepared using a solution for preparing a sample (2A), “stool sample (2B)” refers to a stool sample prepared using a solution for preparing a sample (2B), and “stool sample (2D)” refers to a stool sample prepared using a solution for preparing a sample (2D), respectively.

As a result, with respect to the stool sample (2D), although the presence of amplified PCR products was confirmed when the sample preserved for 1 day was used, no amplification was observed when using the samples preserved for 3 days or longer. On the other hand, with respect to the stool samples (2A) and (2B) prepared using a solution for preparing a sample (2A) or a solution for preparing a sample (2B) which were the solutions for preparing stool samples according to the present invention, the presence of amplified PCR products was confirmed even when the samples preserved for 10 days were used. Meanwhile, with respect to the stool sample (2C) prepared using a solution for preparing a sample (2C) disclosed in Japanese Translation of PCT Application No. 2004-500897, no amplification of PCR products was observed even when using the sample preserved only for 1 day.

From the above results, it is evident that from the stool samples prepared by the preparation method according to the present invention, it is possible to efficiently recover nucleic acids contained in stool. In addition, by using the stool samples according to the present invention, it is also apparent that the accuracy for RNA analysis may also be improved. It is thought that this is because by using the solution for stool sample according to the present invention, the nucleic acids originating from mammalian cells that are contained in the stool and even RNA which is particularly prone to degradation, can be stably preserved for a long time at room temperature.

On the other hand, because no amplification of PCR products originating from the stool sample (2C) was observed, when a solution containing an antibiotic was used as the solution for preparing a sample, although bacterial cells will be killed by the antibiotic, it is possible that the RNA degradation may even be accelerated due to the release of RNase or the like from the dead bacterial cells. In addition, because the number of mammalian cells contained in stool is small, when the mammalian cells are separated from the stool, as compared to the method for recovering nucleic acids according to the present invention in which the nucleic acids originating from bacterial cells may function as a carrier, it is possible that sufficient amount of nucleic acids may be difficult to recover.

### Reference Example 3

Ethanol solutions of 0%, 10%, 20%, 30%, 40%, 50%, 60%, 70%, 80%, 90% and 100% were prepared by dilution using ultra-pure water. 5 mL of each of these ethanol solutions was dispensed into 15-mL polypropylene tube.

After dispensing 0.5 g of stool collected from a healthy individual to each of these tubes, the tubes were left statically at 37° C. for 48 hours. Thereafter, each tube was centrifuged, and the resulting supernatant was removed to obtain a solid component. 3 mL of a phenol mixture “Trizol” (manufactured by Invitrogen Corporation) was added to the obtained solid components, and the samples were sufficiently mixed for 30 seconds or more using a homogenizer, followed by the addition of 3 mL of chloroform. Then, the resultant was centrifuged at 12,000×g for 10 minutes. The supernatant (aqueous layer) obtained as a result of the centrifugation was collected in a new polypropylene tube. Thereafter, RNA was recovered from the collected supernatant using the RNeasy midi kit (manufactured by Qiagen GmbH).

FIG. 8 is a diagram showing the amount of RNA recovered from stool samples prepared using ethanol solutions of each concentration. As a result, it is clear that when an alcohol such as ethanol is used as an active ingredient of the solution for preparing a sample, the alcohol concentration is preferably at least 30%, more preferably at least 50%, still more preferably within a range from 50 to 80%, and most preferably within a range from 60 to 70%.

### Reference Example 4

Stool collected from five healthy individuals was mixed adequately and was then dispensed into two 15-mL polypropylene tubes (0.2 g each). 1 mL of a 32% modified ethanol solution containing 18% of isopropanol (having a total alcohol concentration of 50%) was added to one of the polypropylene tubes and mixed adequately, and the tube was then left statically at 25° C. for 1 day. The prepared stool sample was used as a stool sample (4A). One of the remaining polypropylene tubes was used as a control sample, and was quickly transferred to a deep freezer set at −80° C. after the dispensation.

DNA was recovered from both stool samples using the QIAamp DNA Stool Mini Kit (manufactured by Qiagen GmbH) which was a DNA extraction kit from stool. The concentration of the recovered DNA was quantified by spectrophotometry. As a result, it was possible to recover almost the same amount of DNA from both stool samples.

A mutation analysis was conducted, using 100 ng of the recovered DNA as well as the “K-ras codon 12 mutations detection reagent” (manufactured by Wakunaga Pharmaceutical Co., Ltd.) which was a kit for analyzing mutations in the K-ras gene, and following the protocol attached to the kit. As a result, the analyses of DNA recovered from the stool sample (4A) against 6 types of mutated genes were all negative, as was the case where the DNA recovered from the control sample was used.

From the above results, it is evident that by using the nucleic acids recovered by the method for preparing a stool sample according to the present invention and the method for recovering nucleic acids according to the present invention, even the analyses of nucleic acids which require a high level of accuracy, such as the analyses of gene mutations, can be carried out with an adequate level of accuracy. In addition, although modified ethanol prepared by mixing isopropanol and ethanol was used in the present example as a process solution, equivalent results were obtained even when a 50% ethanol solution which had the same alcohol concentration as that of the modified ethanol was used.

### Reference Example 5

Stool collected from one healthy individual was dispensed into three 15-mL polypropylene tubes (0.1 g each). 3 mL of a 70% ethanol solution was added to one of the polypropylene tubes to sufficiently disperse the stool, and the obtained stool sample was used as a stool sample (10A). On the other hand, to the remaining two polypropylene tubes, 2.4 mL of “ISOGEN” (manufactured by Nippon Gene Co., Ltd.) was each added to sufficiently disperse the stool, and the obtained stool samples were used as comparative samples (P1) and (P2). It should be noted that “ISOGEN” is a phenol-containing material that contains 40% of phenol (having a water solubility of about 10% by weight).

RNA was rapidly recovered from the comparative sample (P1) following the stool dispersion. More specifically, the stool sample was sufficiently mixed for 30 seconds or more using a homogenizer, followed by the addition of 3 mL of chloroform. Then, the resultant was centrifuged at 12,000×g for 10 minutes. The supernatant (aqueous layer) obtained as a result of the centrifugation was collected in a new polypropylene tube. Thereafter, RNA was recovered from the collected supernatant using the RNeasy midi kit (manufactured by Qiagen GmbH).

As for the comparative sample (P2), after statically leaving the sample at room temperature for 5 hours, RNA was recovered from it in the same manner as that described for the comparative sample (P1).

On the other hand, the stool sample (5A) was left statically at room temperature for 5 hours, just like the comparative sample (P2). Then the stool sample (5A) was centrifuged and the resulting supernatant was removed to obtain precipitates (solid components). RNA was recovered in the same manner as that described for the comparative sample (P1), after adding 2.4 mL of “ISOGEN” to the obtained precipitates.

The recovered RNA was quantified using the Nanoprop instrument (manufactured by Nanoprop Technologies, Inc.). As a result, although it was possible to recover 32 μg of RNA from the comparative sample (P1) with which the RNA recovery was conducted immediately after the preparation of stool sample, only 14 μg of RNA was recovered from the comparative sample (P2) with which the RNA recovery operation was conducted after statically leaving the sample at room temperature for 5 hours. On the other hand, from the stool sample (5A), although the RNA recovery operation was conducted after statically leaving the sample at room temperature for 5 hours, it was possible to recover 57 μg of RNA, which was far more than the amount of RNA recovered from the comparative sample (P1).

From these results, it is clear that by using the solution for preparing a sample according to the present invention, RNA may be recovered highly efficiently, as compared to the conventional cases where a phenol solution was used.

### INDUSTRIAL APPLICABILITY

Since the method for preparing a sample of the present invention allows inhibitory actions on an enzyme reaction using a nucleic acid as a substrate in a nucleic acid-containing sample to be decreased, the present invention can be used particularly in fields such as clinical testing, including routine health examinations, using stool samples.

By the method for preparing a sample according to the present invention, it is able to decrease inhibitory actions on an enzyme reaction using a nucleic acid as a substrate in a nucleic acid-containing sample. Consequently, by recovering nucleic acids from a prepared sample prepared by the method for preparing a sample of the present invention and analyzing the recovered nucleic acid, it is able to decrease effects of inhibitory substances and the like, and to obtain analysis results which have higher reliability, better accuracy, and better specificity.

### BRIEF DESCRIPTION OF THE REFERENCE SYMBOLS

1 Container body
1a Protrusion
2 Cover
3 Stool collection rod
3a Cup
S Solution for preparing a sample
11 Container body
12 Cover
13 Stool collection rod
13a Slot
13b Movable cover
15 Pouch
E Stool

### SEQUENCE LISTINGS

PCT International Patent Application No. PCT/JP2009/070171 sequence list

1. A method for preparing a sample, comprising: mixing a nucleic acid-containing sample with a solution for preparing a sample having one or more members selected from the group consisting of a polycation and a chelating agent as an active ingredient,
wherein the prepared sample is used for recovering a nucleic acid.

2. The method for preparing a sample according to claim 1,
wherein the solution for preparing a sample further contains a water-soluble organic solvent as an active ingredient.

3. The method for preparing a sample according to claim 1,
wherein the solution for preparing a sample contains a polylysine as a polycation.

4. The method for preparing a sample according to claim 1,
wherein the solution for preparing a sample contains EDTA as a chelating agent.

5. The method for preparing a sample according to claim 1,
wherein the solution for preparing a sample has a buffering action.

6. The method for preparing a sample according to claim 1,
wherein the pH of the solution for preparing a sample is from 2 to 6.5.

7. The method for preparing a sample according to claim 2,
wherein the water-soluble organic solvent is one or more members selected from the group consisting of a water-soluble alcohol, ketone and aldehyde.

8. The method for preparing a sample according to claim 2,
wherein the water-soluble organic solvent is one or more members selected from the group consisting of a water-soluble alcohol and ketone, and the concentration of the water-soluble organic solvent is 30% or more.

9. The method for preparing a sample according to claim 2,
wherein the water-soluble organic solvent contains one or more members selected from the group consisting of ethanol, propanol and methanol as water-soluble alcohol.

10. The method for preparing a sample according to claim 2,
wherein the water-soluble organic solvent is ethanol.

11. The method for preparing a sample according to claim 2,
wherein the water-soluble organic solvent contains one or more members selected from the group consisting of acetone and methyl ethyl ketone as ketone.

12. The method for preparing a sample according to claim 2,
wherein the water-soluble organic solvent is an aldehyde, and the concentration of the water-soluble organic solvent is within a range of 0.01 to 30%.

13. The method for preparing a sample according to claim 1,
wherein in terms of a mixing ratio of the nucleic acid-containing sample and the solution for preparing a sample, a volume of the solution for preparing the sample is 1 or more relative to 1 volume of the nucleic acid-containing sample.

14. The method for preparing a sample according to claim 1,
wherein the mixture of the nucleic acid-containing sample and the solution for preparing a sample is stored for a predetermined amount of time.

15. The method for preparing a sample according to claim 14,
wherein the amount of time during which the mixture is stored is 1 hour or more.

16. The method for preparing a sample according to claim 14,
wherein the amount of time during which the mixture is stored is 12 hours or more.

17. The method for preparing a sample according to claim 14,
wherein the amount of time during which the mixture is stored is 24 hours or more.

18. The method for preparing a sample according to claim 14,
wherein the amount of time during which the mixture is stored is 72 hours or more.

19. The method for preparing a sample according to claim 16,
wherein the pH of the solution for preparing a sample is from 3 to 6.

20. The method for preparing a sample according to claim 16,
wherein the pH of the solution for preparing a sample is from 4.5 to 5.5.

21. The method for preparing a sample according to claim 1,
wherein the solution for preparing a sample further contains a surface active agent.

22. The method for preparing a sample according to claim 1,
wherein the solution for preparing a sample further contains a colorant.

23. The method for preparing a sample according to claim 1,
wherein the nucleic acid-containing sample is one or more members selected from the group consisting of stool, blood, and urine.

24. The method for preparing a sample according to claim 1,
wherein the nucleic acid-containing sample is stool.

25. A solution for preparing a sample, comprising: one or more members selected from the group consisting of a polycation and a chelating agent as an active ingredient,
wherein the solution is used for recovering a nucleic acid from the nucleic acid-containing sample.

26. The solution for preparing a sample according to claim 25,
wherein the solution for preparing a sample contains a polylysine as a polycation.

27. The solution for preparing a sample according to claim 25,
wherein the solution for preparing a sample contains EDTA as a chelating agent.

28. The solution for preparing a sample according to claim 25,
wherein the solution for preparing a sample further contains a water-soluble organic solvent as an active ingredient.

29. The solution for preparing a sample according to claim 28,
wherein the water-soluble organic solvent is one or more members selected from the group consisting of a water-soluble alcohol, ketone and aldehyde.

30. A stool collection kit, comprising:
a stool collection container; and
a solution for preparing a sample having one or more members selected from the group consisting of a polycation and a chelating agent as an active ingredient,
wherein the stool collection container includes the solution for preparing a sample.

31. A prepared sample prepared by the method for preparing a sample according to claim 1.

32. A method for analyzing a nucleic acid comprising:
(i) preparing a prepared sample by mixing a nucleic acid-containing sample with a solution for preparing a sample having one or more members selected from the group consisting of a polycation and a chelating agent as an active ingredient,
(ii) recovering a nucleic acid from a cell contained in the prepared sample prepared in the step (i), and
(iii) analyzing the nucleic acid recovered in the step (ii).

33. The method for analyzing a nucleic acid according to claim 32,
wherein the step (i) includes:
(i-1) immersing the nucleic acid-containing sample in the solution for preparing a sample, or
(i-2) immersing the nucleic acid-containing sample in the solution for preparing a sample, and mixing to suspend the nucleic acid-containing sample.

34. A method for analyzing a nucleic acid, comprising: conducting reverse transcriptase reaction or nucleic acid chain elongation reaction in a reaction solution containing a polycation.

35. The method for analyzing a nucleic acid according to claim 34,
wherein the reaction solution contains a polylysine as a polycation.

36. A method for recovering nucleic acid from a stool, comprising:
simultaneously recovering a nucleic acid derived from indigenous intestinal bacterium and a nucleic acid derived from an organism other than indigenous intestinal bacterium, from the stool sample, and
the stool sample is prepared by mixing a collected stool with a solution for preparing a sample having one or more members selected from the group consisting of a polycation and a chelating agent as an active ingredient.

37. The method for recovering a nucleic acid from a stool according to claim 36,
wherein the nucleic acid derived from the organism other than indigenous intestinal bacterium is the nucleic acid derived from a mammalian cell.

38. The method for recovering a nucleic acid from a stool according to claim 36,
wherein the method comprising:
(a) denaturing a protein in the stool sample and thereby extracting a nucleic acid from indigenous intestinal bacterium and an organism other than indigenous intestinal bacterium in the stool sample; and
(b) recovering the nucleic acid extracted in the step (a).

39. The method for recovering a nucleic acid from a stool according to claim 38,
further comprising, following the step (a) and prior to the step (b),
(c) removing the protein denatured in the step (a).

40. The method for recovering a nucleic acid from a stool according to claim 38,
wherein denaturing of a protein in the step (a) is carried out using one or more material selected from the group consisting of a chaotropic salt, an organic solvent and a surface active agent.

41. The method for recovering a nucleic acid from a stool according to claim 40,
wherein the organic solvent is phenol.

42. The method for recovering a nucleic acid from a stool according to claim 39,
wherein the removal of denatured protein in the step (c) is carried out using chloroform.

43. The method for recovering a nucleic acid from a stool according to claim 38,
wherein the recovery of nucleic acid in the step (b) includes:
(b1) adsorbing the nucleic acid extracted in the step (a) to an inorganic support, and
(b2) eluting the nucleic acid adsorbed in the step (b1) from the inorganic support.

44. The method for recovering a nucleic acid from a stool according to claim 38,
further comprising, prior to the step (b),
(d) recovering a solid component from the stool sample.

45. A method for analyzing a nucleic acid comprising:
conducting an analysis of a nucleic acid derived from a mammalian cell,
wherein the nucleic acid is recovered from a stool sample by use of the method for recovering a nucleic acid according to claim 36.

46. The method for analyzing a nucleic acid according to claim 45,
wherein the mammalian cell is a gastrointestinal tract cell.

47. The method for analyzing a nucleic acid according to claim 45,
wherein the mammalian cell is a cell exfoliated from a large intestine.

48. The method for analyzing a nucleic acid according to claim 45,
wherein the nucleic acid derived from a mammalian cell is a marker indicating a neoplastic transformation.

49. The method for analyzing a nucleic acid according to claim 45,
wherein the nucleic acid derived from a mammalian cell is a marker indicating an inflammatory gastrointestinal disease.

50. The method for analyzing a nucleic acid according to claim 45,
wherein the nucleic acid derived from a mammalian cell is a nucleic acid derived from COX-2 gene.

51. The method for analyzing a nucleic acid according to claim 45,
wherein the analysis is one or more of RNA analysis and DNA analysis.

52. The method for analyzing a nucleic acid according to claim 51,
wherein the RNA analysis is one or more analysis selected from the group consisting of an analysis for insertion, deletion, substitution, duplication or inversion of one or more bases in the RNA, an analysis for a splicing variant, an mRNA expression analysis, and a functional RNA analysis.

53. The method for analyzing a nucleic acid according to claim 51,
wherein the DNA analysis is one or more of a mutation analysis and an analysis of an epigenetic change.

54. The method for analyzing a nucleic acid according to claim 53,
wherein the mutation analysis is an analysis for one or more mutations of an insertion, deletion, substitution, duplication or inversion of one or more bases.

55. The method for analyzing a nucleic acid according to claim 53,
wherein the analysis of an epigenetic change is one or more of a DNA methylation analysis and a DNA demethylation analysis.

56. The method for analyzing a nucleic acid according to claim 53,
wherein the mutation analysis is a mutation analysis of a K-ras gene.
